(19) United States
(12) Patent Application Publication
(10) Pub. No.: US 2026/0347928 A1
(43) Pub. Date: Nov. 5, 2026

(54) METHODS AND KITS CONFIGURED FOR HIGH-THROUGHPUT INTERROGATION OF NUCLEIC ACID-CONTAINING SAMPLES WITH HIGH DYNAMIC RANGE

(71) Applicant: William Marsh Rice University, Houston, TX (US)

(72) Inventors: Mingjie DAI, Houston, TX (US); Yan YAN, Houston, TX (US)

(73) Assignee: William Marsh Rice University, Houston, TX (US)

(21) Appl. No.: 19/491,087

(22) PCT Filed: Jun. 7, 2024
(86) PCT No.: PCT/US2024/033027

§ 371 Date: Dec. 8, 2025

Related U.S. Application Data
(60) Provisional application No. 63/506,995, filed on Jun. 8, 2023.

(51) Int. Cl. C12Q 1/6869; C12Q 1/48; C12Q 1/6809; C12Q 1/6813; C12Q 1/6851; C12Q 1/686; C12Q 1/6876; C40B 40/06; C40B 50/06; C40B 70/00

(52) U.S. Cl. CPC C12Q 1/6869; C12Q 1/48; C12Q 1/6809; C12Q 1/6813; C12Q 1/6851; C12Q 1/686; C12Q 1/6876; C12Y 207/07; C40B 40/06; C40B 50/06; C40B 70/00; C12Q 2600/16

(57) ABSTRACT

This application describes compositions, methods, and kits for simultaneous, multiplexed detection and quantification of nucleic acid molecules. In particular, compression PCR is disclosed for compressing the dynamic range of highly complex biological samples, and improve the accuracy and efficiency of high-throughput sequencing analysis.

### REFERENCE TO RELATED APPLICATIONS

The present application claims the priority benefit of U.S. provisional application No. 63/506,995, filed Jun. 8, 2023, the entire contents of which are incorporated herein by reference.

### STATEMENT OF FEDERALLY SPONSORED RESEARCH

This invention was made with government support under Grant No. GM140211 awarded by the National Institutes of Health. The government has certain rights in the invention.

### REFERENCE TO A SEQUENCE LISTING

This application contains a Sequence Listing XML, which has been submitted electronically and is hereby incorporated by reference in its entirety. Said Sequence Listing XML, created on Jun. 7, 2024, is named RICEP0139WO_ST26.xml and is 40,755 bytes in size.

### BACKGROUND

### 1. Field

This application relates generally to compositions, methods, kits and apparatuses for carrying out nucleic acid sequence amplification, and more specifically to compositions, methods, kits and apparatuses for detecting and/or quantitating polynucleotide sequences.

### 2. Description of Related Art

Nucleic acid samples with a high or super high dynamic range can be problematic for next-generation sequencing (NGS) or other quantification methods. Dynamic range refers to the difference in concentration between the most abundant and least abundant molecular species in a sample. A high dynamic range means that the levels of certain molecules are significantly higher than others, which can pose challenges in accurate sequencing and data analysis. In NGS, the DNA or RNA is fragmented and sequenced, and then the sequence reads are aligned to a reference genome or transcriptome. However, a small fraction of high-abundance molecular species take over a large fraction of sequencing reads; while the vast majority of low-abundance species only share a tiny fraction of the sequencing reads in the sequencing data, which limits the accuracy and completeness of the analysis.

High dynamic range samples can also require more sequencing depth and coverage to capture the full range of expression levels accurately. This can result in higher sequencing costs, as more reads are required to ensure that the data is reliable and representative of the sample. Moreover, high dynamic range samples can also require more extensive data analysis, including normalization and statistical modeling, to account for the differences in expression levels and identify meaningful patterns and associations.

As such, technologies are in great need that can help reduce the challenge of high dynamic range and improve the accuracy and efficiency of NGS analysis, ultimately saving costs and enabling more comprehensive and meaningful insights into biological quantification, including, e.g., gene expression profiling and cell type analysis.

### SUMMARY

In an aspect, this disclosure provides a method for sequencing a plurality of target sequences in a nucleic acid mixture sample, the method comprising: (a) subjecting the nucleic acid mixture sample to an asymmetric PCR reaction (i) in a multiplexed format for the plurality of target sequences, and (ii) for each individual target sequence, using an excess primer and a limiting primer each comprising at least a sequence portion capable of binding to a separate strand of the individual target sequence, wherein the excess primer is at a concentration at least 5 fold higher than the limiting primer; and (b) preparing a high-throughput sequencing or hybridization library from nucleic acid products amplified from the asymmetric PCR reaction.

In another aspect, this disclosure provides a method for sequencing a plurality of target sequences in a nucleic acid mixture sample, the method comprising: (a) subjecting the nucleic acid mixture sample to an asymmetric PCR reaction in a multiplexed format for the plurality of target sequences, wherein the asymmetric PCR reaction involves three primers for each target sequences: (i) a forward primer, (ii) a reverse primer, and (iii) an excess primer; wherein both the forward and reverse primers are limiting primers, while the excess primer is shared among all or some of the target sequences and is capable to binding to amplicons generated by the forward and reverse primers; and (b) preparing a high-throughput sequencing or hybridization library from nucleic acid products amplified from the asymmetric PCR reaction.

In a further aspect, this disclosure provides a kit comprising a panel of primer pairs, each pair comprising an excess primer and a limiting primer, at a stock concentration configuration pre-determined for asymmetric PCR amplification off a desired target amplicon.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1J. FIG. 1A provides a first exemplary schematic of multiplexed compression PCR (cPCR) workflow. In this exemplary workflow, for each target polynucleotide, the same excess primer is involved in both Stage 1 (exponential amplification stage) and Stage 2 (linear amplification stage). P__{E1} (multiplexed to P__{Ek}) and P__{L1} (multiplexed to P__{Lk}) represent a list of excess and limiting primers, respectively. T_₁ (multiplexed to T_ₖ) represent amplification target sequences which can be either ssDNA or dsDNA, with ssDNA shown as illustration. Pr_₁ (multiplexed to Pr_ₖ) are Taqman probes used for RT-PCR signal readout and are not necessary for general cPCR reaction intended for sequencing readout. The procedure (before library preparation) is performed in two separate reactions. Rxn 1 is an asymmetric reaction with a multitude of primer pairs (and probes, for RT-PCR but not necessary for sequencing-based workflow), Rxn 2 is a single-cycle primer extension reaction that converts ssDNA product from Rxn 1 to dsDNA. FIG. 1B depicts a comparison between an exponential amplification stage (conventional PCR) and a linear amplification stage, and the combination of the two stages in a cPCR. FIG. 1C depicts a schematic of the two-phase reaction in cPCR resulting the different concentrations of both primers. The exemplary schematic illustrates where the limiting primer (P_{L}) is used up after the first, exponential phase, and a prolonged linear amplification proceeds with only the presence of excess primer (P_{E}). FIG. 1D depicts graphs illustrating the expected results of a conventional PCR and a cPCR monitored in real-time, and compressed dynamic range due to a linear amplification phase which transforms the original relative abundance of a nucleic acid species into its logarithm. Ct is the cycle number at which a detectable signal is produced above background. FIG. 1E provides further exemplary schematics of PCR versus cPCR. These comparisons are illustrated through amplification curves and product versus input plot, showing only cPCR can achieve effective dynamic range compression. It is shown here that cPCR allows more accurate quantitation of low-abundance genes. In the cPCR conversion formula, [P_{L}], [T], and [Amp] denote the concentration of limiting primer, target, and cPCR product, respectively. FIG. 1F provides a second exemplary schematic of multiplexed Compression PCR (cPCR) workflow. In this exemplary workflow, for each target polynucleotide, different primers are involved in Stage 1 (exponential amplification stage) and Stage 2 (linear amplification stage). Here, Stage 1 involves two primers (P_{F1} and P_{R1}, till P_{Fk} and P_{Rk}) for each target polynucleotide, each of the two primers contain a target-specific region (P_{Ei} and P_{Li}, respectively) and a common region (P_{E} and P_{C}, respectively). Stage 2 then use a common excess primer P_{E} for the linear amplification of all the target polynucleotides. P_{FA} and P_{RA} represent primers used to generate a high-throughput sequencing library via a primer extension reaction. Different variations of P_{FA} and P_{RA} are contemplated where some may contain barcodes (P_{F-BC} and P_{R-BC}) and others may contain nested primers (P_{FInti} and P_{RInti}). In all cases, if Illumina sequencers are used, different versions of Illumina sequencing primer combinations (P_{F-Seq} and P_{R-Seq}) are contemplated. FIG. 1G provides an exemplary cPCR-based single-cell sequencing workflow for measuring 3′ gene fragment-based expression. Both barcodes (BC) and UMI (universal molecular identifier) are contemplated in this example. Similar sequencing primer combinations (P_{F-Seq} and P_{R-Seq}) are contemplated as in FIG. 1D. FIG. 1H provides another exemplary cPCR-based single-cell sequencing workflow for measuring 5′ gene fragment-based expression. FIG. 1I provides a further exemplary cPCR-based single-cell sequencing workflow for transcriptomics. It can start either before cDNA amplification and/or cleanup, or start from a GEM mixture. FIG. 1J provides schematics and simulated effects of pseudo excess primers. Two exemplary strategies are illustrated here, one with direct pseudo primer (subpanel b, corresponding to FIG. 1A), and the other with pseudo primer applied to an extended common primer sequence (subpanel c, corresponding to FIG. 1D). Both strategies can reduce product concentration relative to primer concentration and can reduce total product concentration in scaling up cPCR reaction to a high level of multiplexing, as illustrated in the figure (85% and 86% reduction are achieved based on simulation).

FIGS. 2A-2B. FIG. 2A depicts qPCR amplification, where traces confirm the two-phase reaction exhibited by cPCR. FIG. 2B demonstrates where cPCR shows a large log-linear dynamic range (5-log).

FIG. 3 shows real-time amplification traces of 4-target multiplex cPCR. Each color indicates a different fluorescence channel.

FIG. 4 depicts real-time PCR fluorescence signal as a function of input target concentration, in 4-target multiplex cPCR. Horizontal axis only applies to the first two targets. Log-linear relationship was observed between signal amplitude and input target concentration.

FIG. 5 shows that the next-generation sequencing read counts appear as a function of input target concentration, in 4-target multiplex cPCR. Horizontal axis only applies to the first two targets. Log-linear relationship was observed between normalized read counts and input target concentration.

FIG. 6 depicts real-time amplification traces of 4-target multiplex cPCR. Each color indicates a different dilution series sample.

FIG. 7 shows that the end-point RT-PCR fluorescence signal appears as a log-linear function of input target concentration, in 4-target multiplex cPCR. Linear signal was observed in low-concentration range with R²=0.997 and 0.987 (5 logs dynamic range).

FIG. 8 shows that the next-generation sequencing read counts (normalized) appear as a log-linear function of input target concentration, in 4-target multiplex cPCR. Linear signal was observed in low-concentration range with R²=0.988 and 0.92.f

FIG. 9 depicts a 4-target multiplexed cPCR test with varying concentrations for #1 and #2, and fixed concentrations for #3 and #4, showing qPCR amplification trace.

FIG. 10 depicts a 4-target multiplexed cPCR test with varying concentrations for #1 and #2, and fixed concentrations for #3 and #4, showing a qPCR end-point signal with high linearity.

FIG. 11 shows that the next-generation sequencing read counts (normalized) appear as a log-linear function of input target concentration, in 8-target multiplex cPCR. Linear signal was observed in range from 10 μM to 100 aM (i.e. 5 logs dynamic range).

FIG. 12 shows that the next-generation sequencing read counts (normalized) appear as a log-linear function of input target concentration, in 8-target multiplex cPCR. Linear signal was observed in range from 10 μM to 100 aM (i.e. 5 logs dynamic range), under appropriately adjusted experimental condition.

FIGS. 13A-13B. FIG. 13A shows that the next-generation sequencing read counts (normalized) appear as a log-linear function of input target concentration, in 96-target multiplex cPCR. Linear signal was observed in range from 10 fM to 100 aM, for 76% (73 out of 96) targets. FIG. 13B, top panel, shows the distribution of R² value from linear fits for all targets, when using forward primer as excess primer (PE=PF) or using reverse primer as excess primer (PE=PR). FIG. 13B, bottom panel, shows the distribution of the better R² value (of the two choices) for all targets.

FIGS. 14A-14B show preliminary data for highly multiplexed (96-target) cPCR test, which high linearity and primer success rate. In FIG. 14A, cPCR testing on a serial dilution sample shows high linearity (R²>0.95 for 75% of primer pairs). FIG. 14B shows accurate detection (+/−25%) on part with RNA-seq.

FIGS. 15A-15B show that the next-generation sequencing read counts (normalized) appear as a function of input target concentration, in 30-target multiplex cPCR applied to human total RNA sample. FIG. 15A shows that the next-generation sequencing read counts against predicted values, after performing linear fits. FIG. 15B shows the difference in next-generation sequencing read between two cPCR experiments shows good linearity against RNA-seq reported differential gene expression, in two human cell line samples (HeLa and Jurkat), observed over a 50,000× dynamic range (of differential expression).

FIG. 16 shows a high barcode coverage by cPCR relative to a standard 10× sequencing dataset.

FIGS. 17A-17E. FIG. 17A depicts exemplary results of a multiplexed single-cell compression sequencing test performed on a hPBMC sample with a panel of 60 gene targets (with PCR-validated primers), designed in three groups (high-, med-, and low-abundance) to span >4 logs of dynamic range as reported on a 10× single-cell profiling test. FIG. 17B shows where cPCR sequencing showed close-to-uniform read allocation among the 60 gene targets compared with 10× Genomics 3′ mRNA assay, despite the underlying large dynamic range, where low abundance genes are enriched by 100-5,000×. FIG. 17C depicts where cPCR sequencing showed 100~5,000× reads improvement compared to 10× Genomics 3′ for low-abundance, achieving a ~1000× reduction in overall dynamic range. FIG. 17D shows that in the mutually detected single cells, particularly for the low-abundance genes, cPCR detected significantly more mapped reads (mapped to same 3′end mRNA location) compared to 10× sequencing. FIG. 17E shows that in the mutually detected single cells, particularly for the low-abundance genes, the map reads covered up to 20× more molecular UMIs and 2-10× more cell barcodes expressing this gene.

FIG. 18 shows a 3-5× higher barcode coverage by cPCR relative standard 10× sequencing dataset for multiplexed single-cell sequencing.

FIG. 19 depicts an exemplary design and experiment workflow for single-cell sequencing with cPCR.

FIGS. 20A-20E. FIG. 20A shows relatively more uniform distribution of sequencing reads across a selected 60-target panel with a wide dynamic range (>3×10⁴). Roughly, from target 1 to target 60, target molecule abundance (i.e., number of molecules or “#mol”) decreases while the cPCR-seq (shown as “IsPCR”) read numbers (per 50 k) remain relatively stable. FIG. 20B shows cPCR allocated ~10× fewer sequencing reads per gene for the most abundant gene group (the right more group), 10~100× more reads for the medium-to-low abundance genes (the middle group), and up to ~1000× more reads for the lowest abundance genes (the left most group). FIG. 20C shows cPCR detects more medium-to-low abundance gene targets compared to standard 10× analysis. FIG. 20D shows, for each medium-to-low abundance gene target, cPCR detected 10-100× more reads relative to standard 10× analysis. FIG. 20E shows, for each medium-to-low abundance gene target, cPCR detected 10-100× more single cells expressing the target gene.

### DETAILED DESCRIPTION

Unless defined otherwise, all technical and scientific terms used herein have the same meaning as commonly understood by one of ordinary skill in the art to which this disclosure belongs.

All publications, patents, and patent applications mentioned in this specification are herein incorporated by reference to the same extent as if each individual publication, patent, or patent application was specifically and individually indicated to be incorporated by reference in their entirety.

As used herein and in the appended claims, the singular forms “a,” “an,” and “the” include plural referents unless the context clearly dictates otherwise. By way of example, “an element” means at least one element and can include more than one element.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc.

The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

### I. GENERAL DEFINITIONS

As used herein, the term “substantially”, when used to modify a quality, generally allows a certain degree of variation without that quality being lost. For example, in certain aspects such degree of variation can be less than 0.1%, about 0.1%, about 0.2%, about 0.3%, about 0.4%, about 0.5%, about 0.6%, about 0.7%, about 0.8%, about 0.9%, about 1%, between 1-2%, between 2-3%, between 3-4%, between 4-5%, or greater than 5% or 10%.

The term “about”, “around” or “approximately”, when modifying the quantity (e.g., mg) of a substance or composition, or the value of a parameter characterizing a step in a method, or the like, refers to variation in the numerical quantity that can occur, for example, through typical measuring, handling, and sampling procedures involved in the preparation, characterization and/or use of the substance or composition; through an inadvertent error in these procedures; through differences in the manufacture, source, or purity of the ingredients employed to make or use the compositions or carry out the procedures; and the like. In certain aspects, “about” can mean a variation of +0.1%, +0.5%, +1%, +2%, +3%, +4%, +5%, +6%, +7%, +8%, +9% or +10%.

As used herein, the term “dynamic range” refers to the difference in concentration between the most abundant and least abundant molecular species in a nucleic acid-containing sample.

The dynamic range of a nucleic acid sample can vary drastically up to 106:1 or even more. Compressing the dynamic range of a nucleic acid sample can be important in high-throughput sequencing applications to increase the sensitivity and accuracy of the sequencing results. Various technologies tried to address the dynamic range issue of a nucleic acid sample via targeted analyses. They include emulsion PCR, hybridization-based methods (e.g., molecular inversion probes (MIPs) and capture probes), size or species selection (e.g., rRNA depletion or poly(A) selection), and digital PCR. None of these methods are satisfactory, especially for compressing the dynamic range of sample in a multi-plex or high-throughput setting.

As used herein, “DNA” refers to deoxyribonucleic acid. DNA can be either single-stranded or double-stranded. DNA typically comprises four nucleotides: cytosine (C), guanine (G), adenine (A), and thymine (T). In an aspect, the sequence of a DNA molecule provided herein comprises one or more degenerate nucleotides. As used herein, a “degenerate nucleotide” refers to a nucleotide that can perform the same function or yield the same output as a structurally different nucleotide. Non-limiting examples of degenerate nucleotides include a C, G, or T nucleotide (B); an A, G, or T nucleotide (D); an A, C, or T nucleotide (H); a G or T nucleotide (K); an A or C nucleotide (M); any nucleotide (N); an A or G nucleotide (R); a G or C nucleotide(S); an A, C, or G nucleotide (V); an A or T nucleotide (W), and a C or T nucleotide (Y).

### II. ASYMMETRIC PCR

Asymmetric PCR is a variation of the traditional PCR technique which amplifies one strand of a double-stranded DNA template, thus producing ssDNA products. In traditional PCR, approximately equal amounts of forward and reverse primers are used, and the amplification proceeds exponentially for both strands of the DNA template. In contrast, in asymmetric PCR, one of the primers is used at a lower concentration compared to the other primer. As a consequence, the exponential amplification stops once the lower primer is used up, and linear amplification continues in which only one primer is extended, generating single-stranded product. See Gyllensten and Erlich, Proc. Natl. Acad. Sci. (USA) 85:7652-7656 (1988); and U.S. Pat. No. 5,066,584.

A modification on the asymmetric PCR, known as Linear-After-The-Exponential-PCR (LATE-PCR) (see U.S. Pat. Nos. 7,632,642 B2; 7,972,786 B2; 9,476,092 B2) uses a limiting primer with a higher melting temperature than the excess primer to maintain reaction efficiency as the lower limiting primer concentration increases the primer melting temperature (Tm). See Sanchez et al. (2004) Proc. Natl. Acad. Sci. (USA) 101:1933-1938; see also, US20060057611A1.

However, both asymmetric PCR and LATE-PCR have not been used as a quantitative assay method or used in combination with high-throughput sequencing. Furthermore, both can result in the formation of non-specific amplification products, which has to be minimized.

Provided herein are methods and compositions for multiplexed asymmetric PCR reactions that effectively reduce the dynamic range of a highly complex nucleic acid sample, and allow for multiplexed quantitative measurement of nucleic acid abundances in the sample. Provided herein are also methods and compositions for improved primer design and optimized reaction conditions that reduce non-specific amplification products and allow for such multiplexed quantitative compression and readout.

### III. COMPRESSION PCR

As used herein, “compression PCR” or “cPCR” refers to a process for treating a complex nucleic acid sample comprising many target sequences (typically up to hundreds, thousands or tens of thousands) and exhibiting a large dynamic range typically more than 100:1 (i.e. 2 logs), and up to 6-8 logs, by compressing the dynamic range through a multiplex asymmetric PCR reaction which performs a log-transform on the target sequences' abundance, thus allowing for efficient and economical detection and quantitation of these targets downstream. The process that performs sequence determination via high-throughput sequencing following cPCR treatment is in particular referred to herein as cPCR-seq (also referenced herein as “IsPCR”).

Typically, the asymmetric PCR conditions of cPCR are designed so that the exponential phase of the reaction terminates before reaching a linear phase. In some embodiments, this can be accomplished by utilizing a limiting concentration of at least one of the primers underlying the exponential phase (referred to as a “limiting primer”). When the limiting primer is consumed by the exponential phase, double-stranded amplicon synthesis terminates. In some embodiments, the amount of limiting primer can be adjusted so that a selected number of exponential amplicons are generated. For example, one of the primers is diluted fivefold to one hundred fold so as to be present in limiting amount of 1-20 percent of the concentration of the excess primer.

Unlike regular PCR which involves the conventional exponential growth of PCR products, compression PCR (cPCR) exhibits a two-phase amplification model. As one illustration, in this model, after the completion of the first, exponential phase, the primer with lower initial concentration (the “limiting primer”, or P_{L}) is depleted and only the primer with higher initial concentration (the “excess primer”, or P_{E}) remains, and the reaction enters a second, linear amplification phase, to produce single-stranded DNA as final product. Without being bound to any scientific theory, the point of transition represents a logarithmic transform of the initial target concentration (effectively a Ct value for RT-PCR tests); whereas the final concentration of the linear amplification (ssDNA product) is linearly related to the number of extra cycles after the transition point. Taken together, the final concentration of the ssDNA product is effectively a logarithmic transform of the original target concentration, and this reaction operates in an unbiased and autonomous way, allowing for effective dynamic range compression of the original sample mixture.

cPCR is advantageous compared to conventional PCR-based target amplification for high-throughput sequencing or hybridization-based quantification, in performing multiplex assays, such as, gene expression analysis. For example, in a conventional PCR, amplification primers are utilized at non-limiting concentrations. Therefore, if the dynamic range of the target sequences is high, e.g., 4-8 logs, the highly expressed genes compete with the lower expressed genes for PCR reagents. As a result, a small fraction of high-abundance genes take over a large fraction of the PCR amplicons; while the vast majority of low-abundance genes only share a tiny fraction of the PCR amplicons and may not be successfully detected during sequencing. In some embodiments, compression PCR avoids this by using a limiting concentration of at least one primer to produce a similar number of double-stranded amplicons for each target sequence which are then linearly amplified. Over-representation of highly expressed genes are thus minimized because the exponential phase amplification is converted to a linear amplification. Therefore, the dynamic range of the amplicons are compressed relative to the original sample, and the over- and under-representation issues of a conventional PCR are avoided. Accordingly, cPCR increases both the sensitivity and accuracy for rare sequence detection.

Disclosed herein are compositions and methods for cPCR. In an aspect, a method is disclosed for sequencing a plurality of target sequences in a nucleic acid mixture sample, the method comprising: (a) subjecting the nucleic acid mixture sample to an asymmetric PCR reaction in a multiplexed format for the plurality of target sequences, using an excess primer and a limiting primer each comprising at least a sequence portion capable of binding to a separate strand of an individual target sequence; and (b) preparing a high-throughput sequencing or hybridization library from nucleic acid products amplified from the asymmetric PCR reaction.

In another aspect, a method is disclosed for sequencing a plurality of target sequences in a nucleic acid mixture sample, the method comprising: (a) subjecting the nucleic acid mixture sample to an asymmetric PCR reaction in a multiplexed format for the plurality of target sequences, wherein the asymmetric PCR reaction involves three primers for each target sequences: (i) a forward primer, (ii) a reverse primer, and (iii) a common excess primer; wherein both the forward and reverse primers are limiting primers, while the common excess primer is shared among all or some of the target sequences and is capable to binding to amplicons generated by the forward and reverse primers; and (b) preparing a high-throughput sequencing or hybridization library from nucleic acid products amplified from the asymmetric PCR reaction.

In another aspect, a cPCR method disclosed herein further comprises converting single-stranded nucleic acids produced from the asymmetric PCR reaction into double-stranded.

In a further aspect, a method disclosed herein further comprises: (c) obtaining sequencing reads data from the high-throughput sequencing library, wherein the sequencing reads data (i) reflect a transformed abundance of the plurality of target sequences in the nucleic acid mixture sample and (ii) exhibit a compressed dynamic range relative to the nucleic acid mixture sample, and (d) determining the original abundance of the plurality of target sequences in the nucleic acid mixture sample based on the transformed abundance.

In another aspect, a cPCR method further comprises: normalizing the sequencing reads data or abundance readout by a normalization factor determined based on at least one of the following four aspects. (i) sequencing yield, i.e. the ratio between the number of sequencing reads and cPCR product concentration (in an aspect, this is done by spiking in a reference amplicon with known sequence at the end of cPCR reaction); (ii) sequence-specific amplification efficiency (in an aspect, this is either measured and tabulated a priori or by spiking in an internal calibration standard with the sample, before starting the cPCR reaction); (iii) functional form of the cPCR transformation, i.e. slope of the log-linear fit (in an aspect, this is either assumed (by formula value), or measured and recorded a priori, or by spiking in internal calibration standard); and (iv) linearity correction of the cPCR transformation. In an aspect, this uses either pre-measured conversion values, or with spiked-in internal standards.

In an aspect, in a method disclosed herein, the excess primer is at a higher concentration than the limiting primer, resulting in preferential amplification of the strand targeted by the excess primer over the strand targeted by the limiting primer.

In an aspect, the length of the spacing between either the positions of two distinct primers or between a primer position and the poly(A) site position may vary. By “spacing” herein is meant the number of nucleotides between two approximate locations. Therefore, the term “spacing” is used herein to define the distance in length between the position of a primer and the position of another primer or a point or a genomic location. Generally, for any given poly(A) site, there may be a particular point or genomic location subsequent the primer where poly(A) may be expected to start. In an aspect, this point or genomic location is more sharply defined. In another aspect, this point or genomic location has a window of, for example, 5, 10, 20, 50, or 100 nucleotides.

In an aspect, the spacing between either the limiting primer position or the excess primer position, and the poly(A) site position is at least a length of 500, 450, 400, 350, 300, 250, 200, 150, 100, 50, 40, 30, or 20 nucleotides. In an aspect, the spacing between either the limiting primer position or the excess primer position, and the poly(A) site position is at most a length of 500, 450, 400, 350, 300, 250, 200, 150, 100, 50, 40, 30, or 20 nucleotides.

In an aspect, the spacing between either the limiting primer position or the excess primer position, and the poly(A) site position is a length of between 500 and 450, between 500 and 400, between 500 and 350, between 500 and 300, between 500 and 250, between 500 and 200, between 500 and 150, between 500 and 100, between 500 and 50, or between 500 and 0 nucleotides. In an aspect, the spacing between either the limiting primer position or the excess primer position, and the poly(A) site position is a length of between 500 and 400, between 450 and 350, between 400 and 300, between 350 and 250, between 300 and 200, between 250 and 150, between 200 and 100, between 150 and 50, or between 100 and 0 nucleotides. In an aspect, the spacing between either the limiting primer position or the excess primer position, and the poly(A) site position is a length of between 500 and 20, between 400 and 20, between 300 and 20, between 200 and 20, between 150 and 20, between 100 and 20, between 90 and 20, between 80 and 20, between 70 and 20, between 60 and 20, between 50 and 20, between 40 and 20, or between 30 and 20 nucleotides.

In an aspect, the spacing between either the limiting primer position and the excess primer position, or the forward primer position and the reverse primer position is at least a length of 500, 450, 400, 350, 300, 250, 200, 150, 100, 50, 40, 30, or 20 nucleotides. In an aspect, the spacing between either the limiting primer position and the excess primer position, or the forward primer position and the reverse primer position is at most a length of 500, 450, 400, 350, 300, 250, 200, 150, 100, 50, 40, 30, or 20 nucleotides.

In an aspect, the spacing between either the limiting primer position and the excess primer position, or the forward primer position and the reverse primer position is a length of between 500 and 450, between 500 and 400, between 500 and 350, between 500 and 300, between 500 and 250, between 500 and 200, between 500 and 150, between 500 and 100, between 500 and 50, or between 500 and 0 nucleotides. In an aspect, the spacing between either the limiting primer position and the excess primer position, or the forward primer position and the reverse primer position is a length of between 500 and 400, between 450 and 350, between 400 and 300, between 350 and 250, between 300 and 200, between 250 and 150, between 200 and 100, between 150 and 50, or between 100 and 0 nucleotides. In an aspect, the spacing between either the limiting primer position and the excess primer position, or the forward primer position and the reverse primer position is a length of between 500 and 20, between 400 and 20, between 300 and 20, between 200 and 20, between 150 and 20, between 100 and 20, between 90 and 20, between 80 and 20, between 70 and 20, between 60 and 20, between 50 and 20, between 40 and 20, or between 30 and 20 nucleotides.

In an aspect, a method disclosed herein further comprises: detecting the nucleic acid products amplified from the asymmetric PCR reaction using a sequencing method, a hybridization method, a microarray method, a digital PCR method, or a quantitative PCR method.

In an aspect, a method disclosed herein is for determining a plurality of target sequences in a nucleic acid mixture sample comprising an original dynamic range of at least 50:1, 100:1, 1000:1, 10000:1, or 100000:1. In another aspect, the plurality of target sequences in the nucleic acid mixture sample comprise an original dynamic range of between 50:1 and 100:1, between 50:1 and 1000:1, between 50:1 and 10000:1, between 100:1 and 500:1, between 500:1 and 1000:1, between 500:1 and 5000:1, between 500:1 and 10000:1, between 1000:1 and 5000:1, or between 1000:1 and 10000:1. The dynamic range of a nucleic acid sample may come from copy number variation of certain target genes and/or differential gene expression levels.

In an aspect, a method disclosed herein log-transforms an original relative abundance into a compressed relative abundance. In another aspect, the original abundance is determined based on the transformed abundance and the concentration of the excess primer used in the asymmetric PCR reaction.

In an aspect, the compressed dynamic range is at or below 2:1, 3:1, 4:1, 5:1, 6:1, 7:1, 8:1, 10:1, 25:1, 50:1, 100:1, or 250:1. In another aspect, the compressed dynamic range is between 2:1 and 3:1, between 2:1 and 4:1, between 2:1 and 5:1, between 5:1 and 10:1, between 5:1 and 15:1, between 5:1 and 20:1, between 10:1 and 50:1, between 10:1 and 100:1, between 50:1 and 100:1, between 50:1 and 250:1, or between 100:1 and 250:1.

In an aspect, a cPCR method disclosed herein is in a multiplexed format of at least 10-plex, 20-plex, 50-plex, 100-plex, 200-plex, 300-plex, 400-plex, 500-plex, 750-plex, 1000-plex, 2000-plex, 5000-plex, 10000-plex, 20000-plex, 50000-plex, or 100000-plex. In another aspect, the multiplexed format is between 500-plex and 1000-plex, between 500-plex and 2000-plex, between 500-plex and 3000-plex, between 500-plex and 4000-plex, between 500-plex and 5000-plex, between 500-plex and 10000-plex, between 1000-plex and 2000-plex, between 2000-plex and 4000-plex, between 2000-plex and 6000-plex, between 2000-plex and 8000-plex, between 5000-plex and 8000-plex, between 5000-plex and 10000-plex, between 10000-plex and 20000-plex, between 20000-plex and 50000-plex, between 50000-plex and 100000-plex.

In an aspect, a cPCR reaction or a cPCR kit provides a single-tube reaction. As used herein a “single-tube” method means a series of at least two operations, for example, sample preparation, amplification or sequencing, that can be performed without transferring the sample from one container, be it a test tube, a reaction well, a chamber in a microfluidics device, a glass slide, or any other apparatus capable of holding a reaction mixture, to another container. In an aspect, a cPCR reaction or a cPCR kit requires reactions in multiple tubes, with optional processing steps in between.

In an aspect, cPCR can be used to compare or quantify polymorphic variants of a particular locus.

Many factors are considered to optimize cPCR conditions, including such as the quantity of each target nucleic acid sequence, the relative amount of each target nucleic acid sequence, the number of different target nucleic acid sequences to be amplified in a single reaction, and the degree of accuracy desired. Further factors to consider include, e.g., sequence design, the type, amount of polymerase used, and polymerase buffer.

In an aspect, a cPCR reaction comprises a series of temperature cycles comprising a denaturation step, an annealing step, and an extension step. In another aspect, a cPCR reaction comprises a series of temperature cycles comprising a denaturation step, and a combined annealing and extension step. In another aspect, the annealing step is at around 55 °C. In another aspect, the annealing step is between 35 and 65, between 40 and 60, between 45 and 55, between 40 and 65, between 45 and 60, between 50 and 60, between 50 and 55, or between 55 and 60 °C.

In an aspect, the extension step is for about 0.5, 1, 2, 3, 4, 5, 6, 7, 8, 9, 10, 11, 12, 13, 15, 18, 20, 30, 40, 60, 75, 90, 105, or 120 minutes. In another aspect, the extension step is for at least 0.5, 1, 2, 3, 4, 5, 6, 7, 8, 9, 10, 11, 12, 13, 15, 18, 20, 30, 40, 60, 75, 90, 105, or 120 minutes. In another aspect, the extension step is for at most 0.5, 1, 2, 3, 4, 5, 6, 7, 8, 9, 10, 11, 12, 13, 15, 18, 20, 30, 40, 60, 75, 90, 105, or 120 minutes.

In an aspect, the annealing step is for about 0.5, 1, 2, 3, 4, 5, 6, 7, 8, 9, 10, 11, 12, 13, 15, 18, 20, 30, 40, 60, 75, 90, 105, or 120 minutes. In another aspect, the annealing step is for at least 0.5, 1, 2, 3, 4, 5, 6, 7, 8, 9, 10, 11, 12, 13, 15, 18, 20, 30, 40, 60, 75, 90, 105, or 120 minutes. In another aspect, the annealing step is for at most 0.5, 1, 2, 3, 4, 5, 6, 7, 8, 9, 10, 11, 12, 13, 15, 18, 20, 30, 40, 60, 75, 90, 105, or 120 minutes.

In an aspect, the annealing/extension combined step is for about 0.5, 1, 2, 3, 4, 5, 6, 7, 8, 9, 10, 11, 12, 13, 15, 18, 20, 30, 40, 60, 75, 90, 105, or 120 minutes. In another aspect, the annealing/extension combined step is for at least 0.5, 1, 2, 3, 4, 5, 6, 7, 8, 9, 10, 11, 12, 13, 15, 18, 20, 30, 40, 60, 75, 90, 105, or 120 minutes. In another aspect, the annealing/extension combined step is for at most 0.5, 1, 2, 3, 4, 5, 6, 7, 8, 9, 10, 11, 12, 13, 15, 18, 20, 30, 40, 60, 75, 90, 105, or 120 minutes.

In a further aspect, the extension step is for between 0.5 and 20, between 1 and 20, between 2 and 20, between 3 and 20, between 4 and 20, between 5 and 20, between 6 and 20, between 7 and 20, between 8 and 20, between 9 and 20, between 10 and 20, between 11 and 20, between 12 and 20, between 13 and 20, between 15 and 20, between 1 and 18, between 2 and 16, between 3 and 14, between 4 and 12, between 5 and 10, between 6 and 9, between 7 and 8, between 4 and 6, between 6 and 8, between 8 and 10, between 9 and 12, or between 10 and 12 minutes.

In an aspect, a cPCR reaction comprises at least about 15, 20, 25, 30, 35, 40, 45, 50, 55, 60, 65, 70, 75, 80, 85 or 90 cycles. In another aspect, a cPCR reaction comprises about 15, 20, 25, 30, 35, 40, 45, 50, 55, 60, 65, 70, 75, 80, 85 or 90 cycles. In a further aspect, a cPCR reaction comprises between 15 and 90, between 20 and 80, between 30 and 70, between 40 and 60, between 25 and 90, between 30 and 90, between 40 and 90, between 50 and 90, between 60 and 90, between 70 and 90, between 25 and 80, between 25 and 70, between 25 and 60, between 25 and 50, between 25 and 40, between 15 and 30, between 25 and 50, or between 35 and 50 cycles, or between 45 and 60 cycles.

### IV. PRIMERS

Primers of this disclosure or useful in methods and kits of this disclosure are oligonucleotides in the broad sense, by which is meant that they may be DNA, RNA, mixtures of DNA and RNA, and they may include non-natural nucleotides (for example, 2′o-methyl ribonucleotides) and non-natural internucleotide linkages (for example, phosphorothioate linkages). Primers function in part by hybridizing to a sequence of interest in a reaction mixture. In an aspect, a primer is a single-stranded oligonucleotide that can hybridize to its complementary sequence at the primer annealing temperature of an amplification reaction and be extended at its 3′ end by a DNA polymerase. In another aspect, a primer of this disclosure can be a primer that signals hybridization of its priming sequence by means of a fluorophore that is indirectly excitable.

In one aspect, primers of this disclosure are short oligonucleotides, generally under fifty bases in length that hybridize to a target strand and are extended by an appropriate polymerase. Although primers are generally linear oligonucleotides, they may include secondary structure. Amplifications often include use of one or more primer pairs each consisting of a forward primer and a reverse primer. In some aspects, in methods, kits and oligonucleotide sets according to this disclosure, either one primer of a pair or both primers of the pair may be labeled with a covalently bound fluorophore that fluoresces when nearby fluorescent DNA dye is stimulated. These primers may be used to monitor synthesis of products resulting by extension of a DNA polymerase such as those resulting from PCR and primer extension assays in real-time or by end-point detection and/or to assess product specificity by melting curve analysis. In an aspect, a primer comprises one or more degenerate nucleotides.

Primers may be target sequence-specific or may be designed to hybridize to sequences that flank a target sequence to be amplified. Thus, the actual nucleotide sequences of each primer may depend upon the target sequence and target polynucleotide, which will be apparent to those of skill in the art. Methods for designing primers suitable for amplifying target sequences of interest are known. See Dieffenbach, C. W. and Dveksler, G. S. (Eds.). (2003). PCR Primer: A Laboratory Manual (2nd ed.). Cold Spring Harbor Laboratory Press.

In an aspect, one or more primer design principles are considered, which in essence include (i) designing primer pool to minimize their 3′-end sequence interactions, (ii) considering the reverse of the P_{L} primer as an “effective primer” during the design and optimization process, (iii) using different Tm/binding energy for P_{E} and P_{L} design.

Generally, a primer should be sufficiently long to prime template-directed synthesis under the conditions of the cPCR reaction. The exact lengths of the primers may depend on many factors, including but not limited to, the desired hybridization temperature between the primers and template polynucleotides, the complexity of the different target polynucleotide sequences to be amplified, the salt concentration, ionic strength, pH and other buffer conditions, and the sequences of the primers and templates. In an aspect, the primers contain from about 15 to about 35 nucleotides that are suitable for hybridizing to a target sequence and form a substrate suitable for DNA synthesis, although the primers may contain more or fewer nucleotides. Shorter primers generally require lower temperatures to form sufficiently stable hybrid complexes with target sequences. The capability of polynucleotides to anneal can be determined by the melting temperature (“Tₘ”) of the hybrid complex. Tₘ is the temperature at which 50% of a polynucleotide strand and its perfect complement form a double-stranded polynucleotide. Therefore, the Tₘ for a selected polynucleotide varies with factors that influence or affect hybridization. In an aspect, in which thermocycling occurs, the amplification primers can be designed to have a melting temperature (“Tₘ”) in the range of about 60-75 °C. Melting temperatures in this range tend to ensure that the primers remain annealed or hybridized to the target polynucleotide at the initiation of primer extension. The actual temperature used for a primer extension reaction may depend upon, among other factors, for example, the concentration of the primers which are used in the cPCR reaction. For amplifications carried out with a thermostable polymerase such as Taq DNA polymerase, in exemplary embodiments the amplification primers can be designed to have a Tₘ in the range of about 60 to about 78 °C or from about 55 to about 70 °C. The melting temperatures of the different amplification primers can be different; however, in an alternative embodiment they should all be approximately the same, i.e., the Tₘ of each amplification primer can be within a range of about 5 °C or less. The Tₘs of various primers can be determined empirically utilizing melting techniques that are well-known in the art. Alternatively, the Tₘ of a primer can be calculated. Numerous references and aids for calculating Tₘs of primers are available in the art and include, by way of example and not limitation, Bresslauer et al., 1986, Proc. Natl. Acad. Sci. USA 83:8893-8897; Freier et al., 1986, Proc. Natl. Acad. Sci. USA 83:9373-9377; Rychlik et al., 1990, Nucleic Acids Res. 18:6409-6412. Any of these methods can be used to determine a Tₘ of a primer. In another aspect, the capability of polynucleotides to anneal can be determined by the binding free energy (ΔG) of the hybrid complex.

In an aspect, a single excess primer is used for one target nucleic acid sequence. In another aspect, multiple excess primers are used for one target nucleic acid sequence. In a further aspect, one excess primer is used for multiple target sequences (e.g. using a poly-T based primer for analyzing mRNA samples). In an aspect, excess or limiting primers are designed to be substantially complementary to regions of the target polynucleotides. By “substantially complementary” herein is meant that the sequences of the primers include enough complementarity, but not complete complementarity, to hybridize to the target polynucleotides at the concentration and under the temperature and conditions employed in the cPCR amplification reaction and to be extended by the DNA polymerase.

In an aspect, a cPCR reaction also uses one or more pseudo excess primers together with their corresponding excess primers. “Pseudo primers” are modified primers that share the same or substantially the same underlying target-binding sequence and include additional modifications such as a polymerase blocker at the 3′ end. Exemplary polymerase blocker includes non-extendable chemical modifications such as inverted dT base, carbon linkers, other non-extendable chemical groups, or non-complementary or non-specific sequences (more can be found in U.S. Pat. No. 11,208,676). Pseudo excess primer compete with their corresponding regular excess primer in target binding, but do not allow polymerase extension. Accordingly, cPCR product concentration would be reduced with the use of pseudo excess primers, which will benefit a high level of multiplexing.

In an aspect, the primers in cPCR may be completely complementary to a target polynucleotide. In another aspect, it may be desirable to include one or more nucleotides of mismatch or non-complementarity in the primers. By “regions of mismatch” and “non-complementarity” are meant a least one nucleotide of a polynucleotide sequence that is not suitable for base-pairing with another polynucleotide sequence. Therefore, the term “region of mismatch” is used when comparing sequences, such as, a primer sequence and a target sequence; a probe sequence and a target sequence; a primer sequence and an amplicon sequence; and the like. In some embodiments, a primer sequence that is a region of mismatch in comparison to a target sequence is substantially unique to that primer. In other embodiments, a primer sequence that is a region of mismatch in comparison to a target sequence also occurs in other primers or probes. Therefore, in some embodiments, a region of mismatch between a primer and a target sequence is a code sequence. By “code sequence” is meant a primer sequence of continuous nucleotides that are not substantially complementary to a target sequence and is substantially unique to that primer. By “substantially unique” is meant the sequence is suitable to identify or distinguish the primer and the amplification products of the primer from other primers and other amplification products. Primers and methods for amplifying sequences to include such code sequence are known in the art (see, e.g., U.S. Pat. Nos. 6,090,552, 6,355,431).

In some embodiments, a region of mismatch between a primer and a target sequence is a sequence that is shared by more than one primer sequence. In various exemplary embodiments, a “shared sequence” may be common to each forward primer, each reverse primer, each excess primer or each limiting primer.

Thus, by “common excess primer” is meant a primer sequence of continuous nucleotides that does not directly bind to a target sequence but is shared by one or more (or each) forward or reverse primer in a cPCR reaction. In an aspect, multiple excess primers are used for multiple target sequences, and each excess primer has an extension that can be further amplified by a unified common excess primer. In another aspect, a number of different common excess primers are used, e.g. each common excess primer corresponds to about 100 (which can any number between 2 and 10,000) target sequences.

Thus by “limiting primer” and “excess primer” may also be meant “forward primer” and “reverse primer” to depict a limiting primer and excess primer with direction orientation.

Determining the number, type, length and composition of regions of mismatch and their position within a primer or probe and their distribution or commonality among the nucleic acids of a cPCR reaction are within the capabilities of the ordinary skilled artisan. Generally, regions of mismatch are designed to perform a user-selected function when incorporated into exponential or linear amplicons. In an aspect, these regions of mismatch serve as excess primer binding sites. In another aspect, the incorporated sequences provide useful sites for downstream hybridization or amplification reactions.

In an aspect, an excess primer is at a concentration at least 2, 3, 4, 5, 6, 7, 8, 9, 10, 11, 15, 20, 25, 30, 35, 40, 45, 50, 75, 100, 150, 200 or 500 fold higher than a corresponding limiting primer. In another aspect, an excess primer is at a concentration between 2 and 5, between 2 and 10, between 5 and 10, between 5 and 15, between 10 and 16, between 10 and 20, between 15 and 30, between 20 and 30, between 25 and 50, between 30 and 40, between 30 and 50, between 30 and 60, between 30 and 70, between 40 and 50, between 40 and 60, between 40 and 70, between 40 and 80, between 50 and 60, between 50 and 100, between 50 and 150, between 70 and 200, or between 100 and 200 fold higher than a corresponding limiting primer.

In an aspect, an excess primer is at a concentration about 20-50, 30-60, 40-80, or 50-100 fold higher than a corresponding limiting primer. In another aspect, an excess primer is at a concentration about 1, 2, 5, 10, 20, 30, 40, 50, 100, 150, 200, 250, 300, 350, 400, 450, 500, 600, 700, 800, 900 or 1000 nM. In another aspect, an excess primer is at a concentration about 1, 2, 5, 10, 20, 30, 40, or 50 μM. In an aspect, an excess primer is at a concentration at least 1, 2, 5, 10, 20, 30, 40, 50, 100, 150, 200, 250, 300, 350, 400, 450, 500, 600, 700, 800, 900 or 1000 nM. In another aspect, an excess primer is at a concentration at least 1, 2, 5, 10, 20, 30, 40, or 50 μM. In an aspect, each of the excess primer concentrations or concentration ranges (or concentration fold difference relative to limiting primers) mentioned in this application (including this paragraph and all preceding and following paragraphs) represents the total concentration of both an excess primer and its corresponding pseudo excess primer(s).

In another aspect, an excess primer is at a concentration between 50 and 1000, between 50 and 900, between 50 and 800, between 50 and 700, between 50 and 600, between 50 and 500, between 50 and 400, between 50 and 300, between 50 and 200, between 50 and 100, between 100 and 1000, between 150 and 1000, between 250 and 1000, between 350 and 1000, between 450 and 1000, between 550 and 1000, between 650 and 1000, between 750 and 1000, between 850 and 1000, between 100 and 900, between 150 and 800, between 250 and 700, between 350 and 600, between 450 and 500, between 150 and 250, between 250 and 350, between 350 and 450, between 450 and 550, between 550 and 650, between 650 and 750, or between 750 and 850 nM.

In another aspect, an excess primer is at a concentration between 0.1 and 0.5, between 0.1 and 0.8, between 0.1 and 1, between 0.5 and 1, between 1 and 5, between 5 and 10, between 10 and 15, between 15 and 25, between 25 and 35, between 35 and 45, between 45 and 55, between 55 and 65, between 65 and 75, between 75 and 85, between 95 and 105, between 105 and 125, between 125 and 155, between 155 and 175, between 175 and 195, or between 195 and 225 nM.

In a further aspect, an amplicon-specific excess primer is at a concentration between 1 and 1000, between 1 and 750, between 1 and 500, between 1 and 250, between 1 and 100, between 1 and 90, between 1 and 80, between 1 and 70, between 1 and 60, between 1 and 50, between 1 and 40, between 1 and 30, between 1 and 20, between 1 and 10, between 1 and 5, between 5 and 1000, between 5 and 750, between 5 and 500, between 5 and 250, between 5 and 100, between 5 and 90, between 5 and 80, between 5 and 70, between 5 and 60, between 5 and 50, between 5 and 40, between 5 and 30, between 5 and 20, between 10 and 95, between 15 and 85, between 25 and 75, between 35 and 65, between 45 and 55, between 15 and 100, between 25 and 100, between 35 and 100, between 45 and 100, between 55 and 100, between 65 and 100, between 75 and 100, between 85 and 100, between 100 and 1000, between 200 and 1000, between 300 and 1000, between 400 and 1000, between 500 and 1000, between 600 and 1000, between 700 and 1000, between 800 and 1000, between 900 and 1000 nM.

In a further aspect, an excess primer or a common excess primer is at a concentration between 0.1 and 0.25, between 0.25 and 0.5, between 0.5 and 1, between 1 and 1.5, between 1.5 and 2.5, between 2.5 and 3.5, between 3.5 and 4.5, between 4.5 and 5.5 between 1 and 5, between 5 and 10, between 10 and 15, between 15 and 25, between 25 and 35, between 35 and 45, or between 45 and 55 μM.

In a further aspect, an excess primer or a common excess primer is at a concentration between 0.1 and 1, between 0.25 and 1, between 0.5 and 5, between 1 and 5, between 1.5 and 5, between 2.5 and 5, between 3.5 and 5, between 4.5 and 15, between 1 and 15, between 5 and 15, between 10 and 25, between 15 and 35, between 25 and 45, or between 35 and 55 μM.

In a further aspect, an excess primer or a common excess primer is at a concentration between 0.1 and 5, between 0.25 and 5, between 0.75 and 5, between 1 and 15, between 1.5 and 15, between 2.5 and 15, between 3.5 and 15, between 4.5 and 25, between 10 and 25, between 15 and 25, between 10 and 35, between 15 and 45, between 25 and 55, or between 15 and 55 μM.

In an aspect, a limiting primer is at a concentration about 0.01, 0.02, 0.03, 0.04, 0.05, 0.1, 0.2, 0.3, 0.4, 0.5, 0.75, 1, 1.5, 2, 2.5, 3, 4, 5, 6, 7, 8, 9, 10, 12.5, 15, 20, 30, 40 or 50, 60, 70, 80, 90, or 100 nM. In another aspect, a limiting primer is at a concentration at least 0.01, 0.02, 0.03, 0.04, 0.05, 0.1, 0.2, 0.3, 0.4, 0.5, 0.75, 1, 1.5, 2, 2.5, 3, 4, 5, 6, 7, 8, 9, 10, 12.5, 15, 20, 30, 40, 50, 60, 70, 80, 90, or 100 nM.

In an aspect, a limiting primer is at a concentration between 0.01 and 0.05, between 0.01 and 0.1, between 0.01 and 0.5, between 0.01 and 1, between 0.01 and 2.5, between 0.01 and 5, between 0.01 and 10, between 0.01 and 20, between 0.01 and 30, between 0.01 and 40, between 0.01 and 50, between 0.01 and 60, between 0.01 and 70, between 0.01 and 80, between 0.01 and 90, between 0.01 and 100, between 0.1 and 1, between 0.1 and 2.5, between 0.1 and 5, between 0.1 and 10, between 0.1 and 20, between 0.1 and 50, between 0.1 and 100, between 1 and 2.5, between 1 and 5, between 1 and 10, between 1 and 20, between 1 and 50, between 1 and 100, between 2.5 and 5, between 2.5 and 10, between 2.5 and 20, between 2.5 and 50, or between 2.5 and 100 nM.

In an aspect, a limiting primer is at a concentration between 0.01 and 45, between 0.1 and 40, between 0.5 and 35, between 1 and 30, between 2.5 and 25, between 5 and 20, between 7.5 and 17.5, between 10 and 15, between 5 and 100, between 5 and 90, between 5 and 80, between 5 and 70, between 5 and 60, between 5 and 50, between 5 and 40, between 5 and 30, between 5 and 20, between 5 and 10, between 10 and 100, between 15 and 100, between 25 and 100, between 35 and 100, between 45 and 100, between 55 and 100, between 65 and 100, between 75 and 100, between 85 and 100, between 10 and 90, between 15 and 80, between 25 and 70, between 35 and 60, between 45 and 50, between 15 and 25, between 25 and 35, between 35 and 45, between 45 and 55, between 55 and 65, between 65 and 75, between 75 and 85, between 85 and 95, or between 95 and 100 nM.

In an aspect, a limiting primer is at a concentration between 2.5 and 25, between 5 and 20, between 7.5 and 17.5, between 10 and 15, between 5 and 100, between 5 and 90, between 5 and 80, between 5 and 70, between 5 and 60, between 5 and 50, between 5 and 40, between 5 and 30, between 5 and 20, between 5 and 10, between 10 and 100, between 15 and 100, between 25 and 100, between 35 and 100, between 45 and 100, between 55 and 100, between 65 and 100, between 75 and 100, between 85 and 100, between 10 and 90, between 15 and 80, between 25 and 70, between 35 and 60, between 45 and 50, between 15 and 25, between 25 and 35, between 35 and 45, between 45 and 55, between 55 and 65, between 65 and 75, between 75 and 85, between 85 and 95, or between 95 and 100 nM.

In an aspect, the concentration differences of an excess primer and a limiting primer are optimized for each target sequence.

In an aspect, an excess primer and a limiting primer have different melting temperatures, and the annealing temperature used in a cPCR reaction is optimized for the excess primer. In another aspect, the annealing temperature used in a cPCR reaction is optimized for both excess and limiting primers. In a further aspect, the optimization of a cPCR condition considers lower concentration of limiting primer(s) which results in different effective ΔG (or Tm) relative to a regular PCR.

### V. TARGET SEQUENCES

As disclosed herein, cPCR is suitable for various types of nucleic acid samples and target sequences, including, e.g., genomic DNA, RNA, cDNA, microRNA, chromatin immunoprecipitation samples, and bisulfite-treated DNA. In an aspect, the nucleic acid samples and target sequences are DNA (e.g., cDNA, genomic DNA or extrachromosomal DNA) or RNA (e.g., mRNA, rRNA or genomic RNA) in nature. The target nucleic acids may be derived or obtained from virtually any sample or source, wherein the sample may optionally be scarce or of a limited quantity. For example, the sample may be one or a few cells collected from a crime scene or a small amount of tissue collected via biopsy. In an aspect, a target nucleic acid sample is from circulating tumor DNA (ctDNA). In another aspect, target nucleic acids may be a synthetic polynucleotide comprising nucleotide analogs or mimics produced for purposes, such as, diagnosis, testing, or treatment.

In an aspect, cPCR is used in a range of gene expression panels for various cancers, including panels for breast, lung, colon, liver, kidney, skin, ovarian and prostate cancer. In another aspect, cPCR is used in a range of gene expression panels for various immune diseases and/or neurological disorders. In an aspect, the nucleic acid mixture sample for cPCR is barcoded for cell origin. In another aspect, a high-throughput sequencing library of cPCR is barcoded with unique molecular identifiers (UMIs).

In an aspect, the nucleic acid products amplified from an asymmetric PCR reaction differ in their length for individual target sequences. In another aspect, the nucleic acid products amplified from an asymmetric PCR reaction have a substantially similar amplicon length. In an aspect, the nucleic acid products amplified from an asymmetric PCR reaction have an amplicon length varying by less than 70%, 60%, 50%, 40%, 30%, 20%, 10% or 5%. In another aspect, the nucleic acid products amplified from an asymmetric PCR reaction have a length of at least 600, 500, 400, 300, 200, 100, 50, 40, 30 or 20 nucleotides. In an aspect, the nucleic acid products amplified from an asymmetric PCR reaction have a length of at most 600, 500, 400, 300, 200, 100, 50, 40, 30 or 20 nucleotides.

In another aspect, the nucleic acid products amplified from an asymmetric PCR reaction have a length of between 600 and 500, between 600 and 400, between 600 and 300, between 600 and 200, between 600 and 100, between 600 and 50, between 600 and 40, between 600 and 30, or between 600 and 20 nucleotides. In an aspect, the nucleic acid products amplified from an asymmetric PCR reaction have a length of between 600 and 500, between 500 and 400, between 400 and 300, between 300 and 200, between 200 and 100, between 100 and 50, between 100 and 40, between 100 and 30, or between 100 and 20 nucleotides. In another aspect, the nucleic acid products amplified from an asymmetric PCR reaction have a length of between 500 and 20, between 400 and 20, between 300 and 20, between 200 and 20, between 150 and 20, between 100 and 20, between 90 and 20, between 80 and 20, between 70 and 20, between 60 and 20, between 50 and 20, between 40 and 20, or between 30 and 20 nucleotides.

### VI. POLYMERASE

As used herein, a “DNA polymerase” refers to an enzyme that is capable of catalyzing the synthesis of a DNA molecule from nucleoside triphosphates. DNA polymerases add a nucleotide to the 3′ end of a DNA strand one nucleotide at a time, creating an antiparallel DNA strand as compared to a template DNA strand. DNA polymerases are unable to begin a new DNA molecule de novo; they require a primer to which it can add a first new nucleotide.

Compression PCR may be carried out with a variety of different DNA polymerases, including for example, Taq, Phusion, Pfu, Q5, AccuPrime Taq, KOD or Vent polymerases. In some embodiments, the DNA polymerase also has 5′-3′ endonuclease activity. In some embodiments, the DNA polymerase is a thermostable polymerase. In some embodiments, the DNA polymerase polymerase has 5′-3′ nuclease activity. Non-limiting examples of polymerases with 5′-3′ nuclease activity include, but are not limited to, AmpliTaq® DNA polymerase, Ampli-Taq® GOLD polymerase and Tth polymerases (Applied Biosystems, Foster City, Calif.), E. coli DNA polymerase I (New England Biolabs, Beverly, Mass.), rBst DNA Polymerase (Epicenter®, Madison, Wis.), and Tfl DNA polymerase (Promega Corp., Madison, Wis.). Moreover, cPCR amplification reactions may be carried out with a variety of different reverse transcriptases, although in some embodiments thermostable reverse-transcriptases are preferred. Suitable thermostable reverse transcriptases include, but are not limited to, reverse transcriptases such as AMV reverse transcriptase, MuLV, and Tth reverse transcriptase. Temperatures suitable for carrying out the various denaturation, annealing and primer extension reactions with the polymerases and reverse transcriptases are well-known in the art. Optional reagents commonly employed in conventional PCR and RT-PCR amplification reactions, such as reagents designed to enhance PCR, modify Tm, or reduce primer-dimer formation, may also be employed in the log-linear amplification reactions.

In an aspect, a DNA polymerase is a thermostable DNA polymerase. As used herein, a “thermostable DNA polymerase” refers to DNA polymerases that can function at high temperatures (e.g., greater than 65 °C) and can survive higher temperatures (e.g., up to about 100 °C). Thermostable DNA polymerases often have maximal catalytic activity at temperatures between 70 °C and 80 °C. In an aspect, a thermostable DNA polymerase is selected from the group consisting of comprising Taq DNA polymerase, Phusion® DNA polymerase, Q5® DNA polymerase, and KAPA High Fidelity DNA polymerase.

In an aspect, a DNA polymerase is a non-thermostable DNA polymerase. As used herein, a “non-thermostable DNA polymerase” refers to DNA polymerases that cannot function at high temperatures. In an aspect, a non-thermostable DNA polymerase is selected from the group consisting of phi29 DNA polymerase and Bst DNA polymerase.

### VII. NGS LIBRARY PREPARATION

In an aspect, a method comprises high-throughput sequencing. In an aspect, a method comprises subjecting a plurality of amplicons to high-throughput sequencing. As used herein, “high-throughput sequencing” refers to any sequences method that is capable of sequencing multiple (e.g., tens, hundreds, thousands, millions, hundreds of millions, tens of billions) DNA molecules in parallel. In an aspect, Sanger sequencing is not high-throughput sequencing. In an aspect, high-throughput DNA sequencing comprises sequencing-by-synthesis or nanopore-based sequencing. In an aspect, high-throughput sequencing comprises the use of a sequencing-by-synthesis (SBS) flow cell. In an aspect, an SBS flow cell is selected from the group consisting of an Illumina SBS flow cell and a Pacific Biosciences (PacBio) SBS flow cell. In an aspect, high-throughput sequencing is performed via electrical current measurements in conjunction with an Oxford nanopore.

Typical preparation of a high-throughput sequencing library is a multistep process that optionally involves sample preparation, fragmentation, end repair and A-tailing, adapter ligation, size selection and PCR amplification, and library quantification and quality control. In cPCR-seq, the ssDNA product from cPCR is first converted to dsDNA before the usual high-throughput sequencing library preparation workflow. In an aspect, the preparation of a high-throughput sequencing library from the nucleic acid products amplified from a cPCR reaction involves adapter ligation. In another aspect, appropriate adapter sequences are embedded (i) within excess primers and/or limiting primers or (ii) within separate adaptor sequences after cPCR reaction and as part of the sequence library preparation workflow.

Typically, high-throughput sequencing generates a sequence file. As used herein, a “sequence file” refers to a computer-readable text file that comprises the sequence of at least one next generation sequencing (NGS) read. As used herein, an “NGS read” refers to a nucleotide sequence of a single nucleic acid molecule generated via a high-throughput sequencing method. In an aspect, an NGS read comprises a UMI sequence. In an aspect, an NGS read comprises a cell barcode sequence. In an aspect, an NGS read comprises a gene sequence. In an aspect, an NGS read comprises a UMI sequence and a gene sequence. In an aspect, an NGS read comprises at least 10 nucleotides. In an aspect, an NGS read comprises at least 25 nucleotides. In an aspect, an NGS read comprises at least 50 nucleotides. In an aspect, an NGS read comprises at least 100 nucleotides. In an aspect, an NGS read comprises at least 250 nucleotides. In an aspect, an NGS read comprises at least 500 nucleotides. In an aspect, an NGS read comprises at least 1000 nucleotides. In an aspect, an NGS read comprises between 10 and 10,000 nucleotides. In an aspect, an NGS read comprises between 10 and 1000 nucleotides. In an aspect, an NGS read comprises between 25 and 150 nucleotides.

In an aspect, a cPCR NGS read comprises no more than 600, 500, 400, 300, 200, 100, 50, 40, 30 or 20 nucleotides. In an aspect, a cPCR NGS read comprises no more than 600, 500, 400, 300, 200, 100, 50, 40, 30 or 20 nucleotides.

### VIII. KIT AND REAGENTS

This disclosure also provides a kit comprising necessary or key reagents for conducting cPCR. Further provided here is a kit comprising a panel of primer pairs, each pair comprising an excess primer and a limiting primer, at a stock concentration configuration pre-determined for cPCR amplification off a desired target amplicon. In an aspect, the panel comprises at least 10, 20, 50, 100, 200, 300, 400, 500, 750, 1000, 2000, 5000, 10000, 20000, or 100000 primer pairs. In an aspect, a cPCR kit comprises a primer panel which comprises one unique limiting primer for each target amplicon while share excess primers across all or subsets of target amplicons. In another aspect, the excess primer and the limiting primer of each primer pair is packed separately. In a further aspect, the primer pairs are individually pre-mixed. In one aspect, all limiting primers have similar concentrations; in another, all limiting primers have different concentrations pre-determined for each amplicon. In another aspect, a cPCR kit further comprises one or more of the following: a set of dNTPs, a polymerase, a buffer solution, and a set of labeled probes.

As used herein “kit” means a collection of reagents for performing an amplification or assay. A kit may be “complete”, that is, include all reagents needed for all steps of an amplification or amplification-detection. Alternatively, a kit may be “partial”, omitting certain reagents needed for those operations. Both complete and partial kits of this disclosure may additionally include reagents for sample preparation, such as nucleic acid isolation and reverse transcription. Sequencing may involve two kits, for example, a complete cPCR amplification kit and a complete sequencing library preparation kit, or the two may be combined into a single kit.

In an aspect, this disclosure provides reagents and buffers needed for cPCR. Non-limiting examples of reagents and buffers needed include Tris-HCl, potassium chloride, magnesium chloride, oligonucleotide primers, deoxynucleotides (dNTPs), DNA polymerases, betaine, and dimethyl sulfoxide. Those of ordinary skill in the art recognize that different DNA polymerases and different target sequences can require different groupings of necessary reagents and buffers.

### IX. EXEMPLARY APPLICATIONS

The compositions and methods disclosed herein are suitable for the detection of gene copy number and/or chromosome copy number in a multiplexed reaction. Further, the methods, assays and kits described herein are applicable for the identification, diagnosing, and monitoring of disorders including, but not limited to cancer, developmental and degenerative disease, neurological disorders, and stem cell disorders.

It will be readily apparent to those skilled in the art that other suitable modifications and adaptations of the devices, systems and methods described herein may be made using suitable equivalents without departing from the scope of the aspects disclosed herein. Having now described certain aspects in detail, the same will be more clearly understood by reference to the following example, which is included for purposes of illustration only and is not intended to be limiting. All patents, patent applications, and references described herein are incorporated by reference in their entirety for all purposes.

### X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—cPCR Workflow Strategies and Expected Results

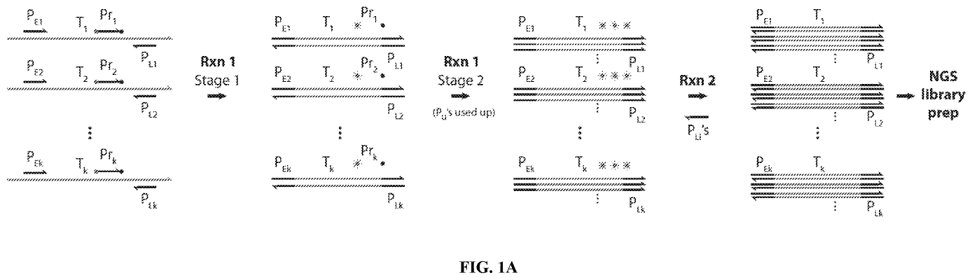

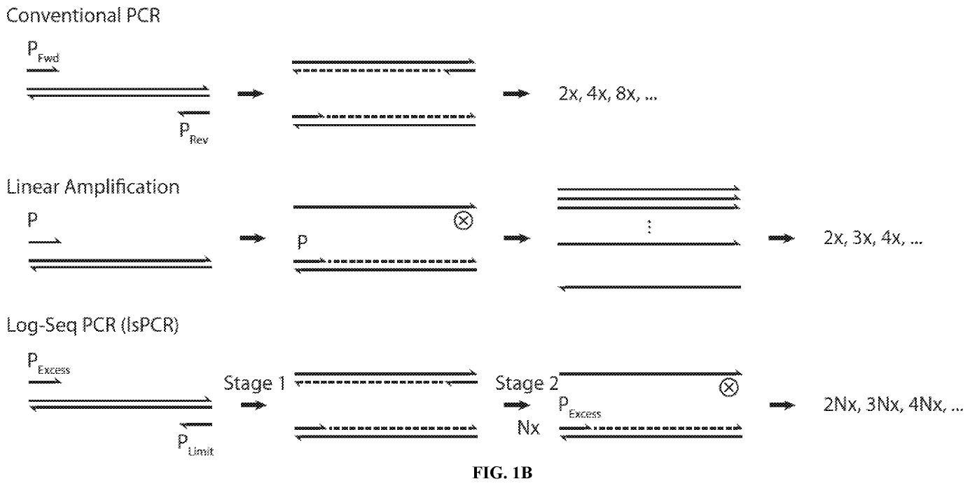

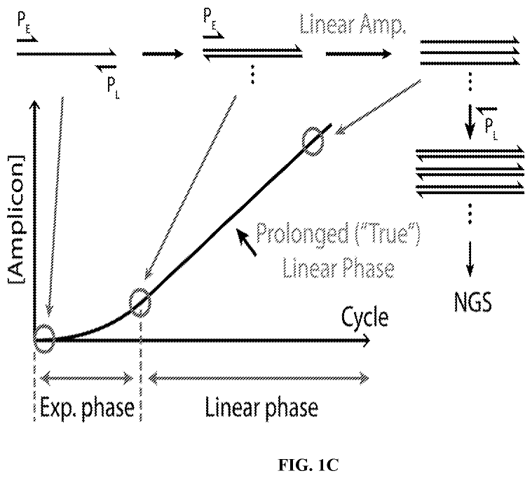

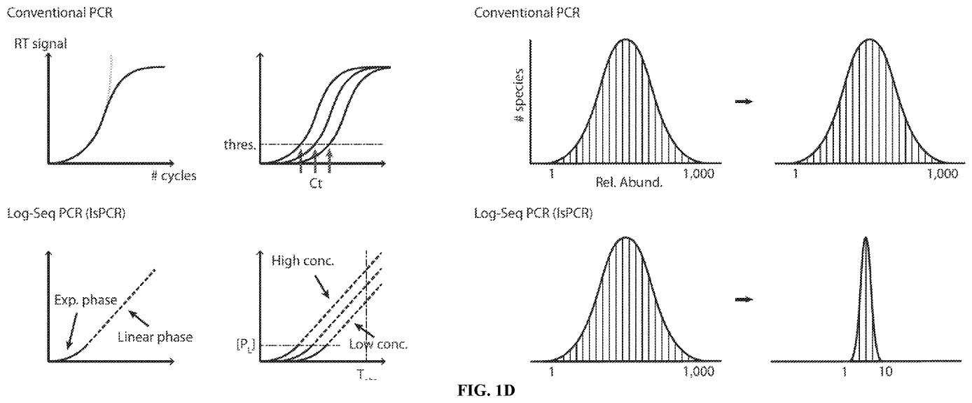

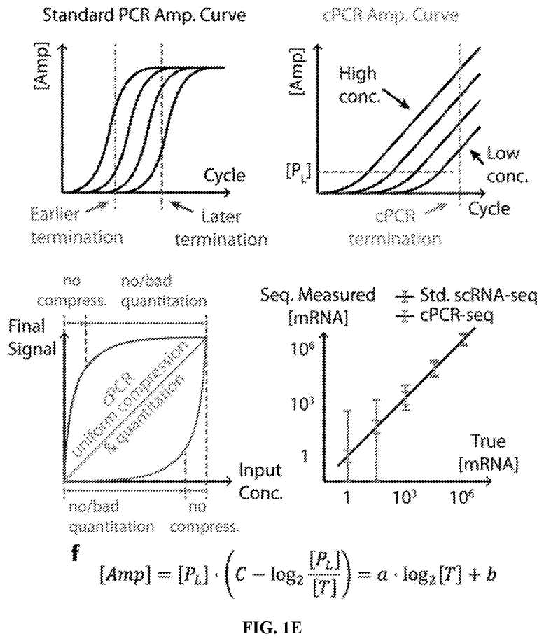

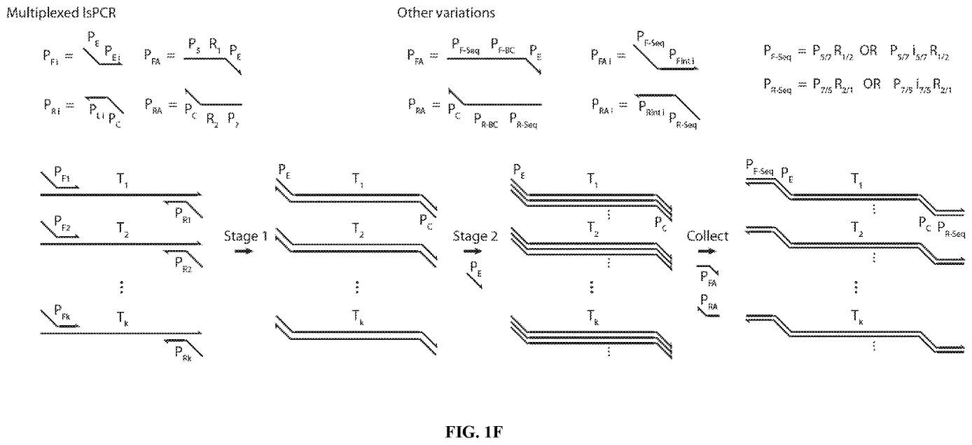

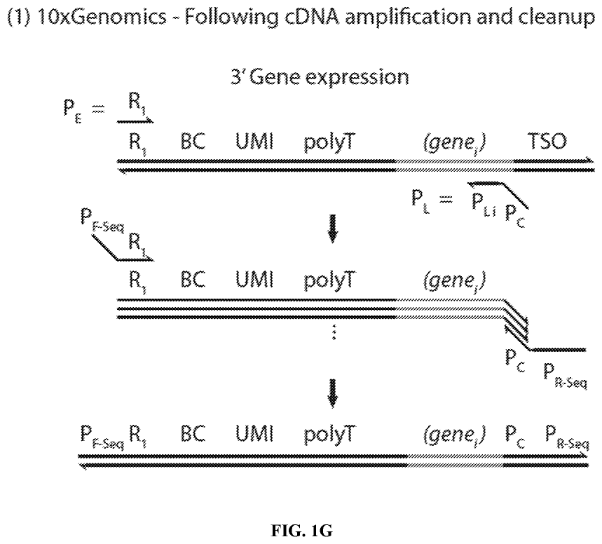

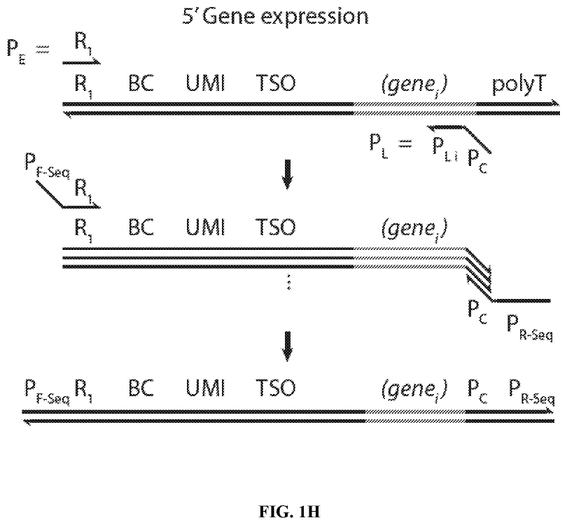

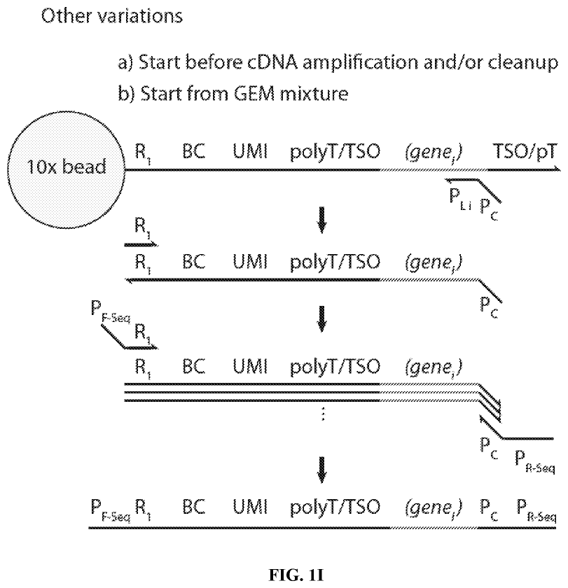

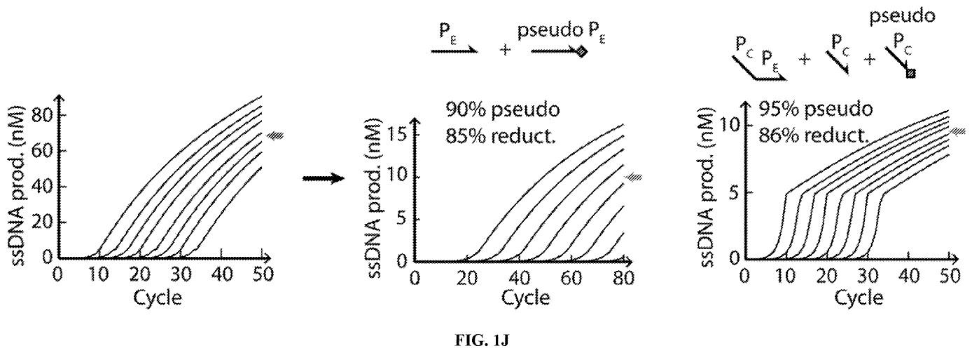

An exemplary typical workflow for compression PCR is illustrated in FIG. 1A. FIG. 1B depicts a comparison between an exponential amplification stage (conventional PCR) and a linear amplification stage, and the combination of the two stages in a cPCR. FIG. 1C depicts a schematic of the two-phase reaction in cPCR resulting the different concentrations of both primers. The exemplary schematic illustrates where the limiting primer (P_{L}) is used up after the first, exponential phase, and a prolonged linear amplification proceeds with only the presence of excess primer (P_{E}). FIG. 1D depicts graphs illustrating the expected results of a conventional PCR and a cPCR monitored in real-time, and compressed dynamic range due to a linear amplification phase which transforms the original relative abundance of a nucleic acid species into its logarithm. FIG. 1E provides further exemplary schematics of PCR versus cPCR. These comparisons are illustrated through amplification curves and product versus input plot, showing only cPCR can achieve effective dynamic range compression. It is shown here that cPCR allows more accurate quantitation of low-abundance genes. In the cPCR conversion formula, [P_{L}], [T], and [Amp] denote the concentration of limiting primer, target, and cPCR product, respectively.

FIG. 1F provides a second exemplary schematic of multiplexed Compression PCR (cPCR) workflow. In this exemplary workflow, for each target polynucleotide, different primers are involved in Stage 1 (exponential amplification stage) and Stage 2 (linear amplification stage). Here, Stage 1 involves two primers (P_{F1} and P_{R1}, till P_{Fk} and P_{Rk}) for each target polynucleotide, each of the two primers contain a target-specific region (P_{Ei} and P_{Li}, respectively) and a common region (P_{E} and P_{C}, respectively). Stage 2 then uses a common excess primer P_{E} for the linear amplification of all the target polynucleotides. P_{FA} and P_{RA} represent primers used to generate a high-throughput sequencing library via a primer extension reaction. Different variations of P_{FA} and P_{RA} are contemplated where some may contain barcodes (P_{F-BC} and P_{R-BC}) and others may contain nested primers (P_{FInti} and P_{RInti}). In all cases, if Illumina sequencers are used, different versions of Illumina sequencing primer combinations (P_{F-Seq} and P_{R-Seq}) are contemplated.

FIG. 1G provides an exemplary cPCR-based single-cell sequencing workflow for measuring 3′ gene fragment-based expression. Both barcodes (BC) and UMI (universal molecular identifier) are contemplated in this example. Similar sequencing primer combinations (P_{F-Seq} and P_{R-Seq}) are contemplated as in FIG. 1D.

FIG. 1H provides another exemplary cPCR-based single-cell sequencing workflow for measuring 5′ gene fragment-based expression. FIG. 1I provides a further exemplary cPCR-based single-cell sequencing workflow for transcriptomics. It can start either before cDNA amplification and/or cleanup, or start from a GEM mixture.

FIG. 1J provides schematics and simulated effects of pseudo excess primers. Two exemplary strategies are illustrated here, one with direct pseudo primer (subpanel b, corresponding to FIG. 1A), and the other with pseudo primer applied to an extended common primer sequence (subpanel c, corresponding to FIG. 1D). Both strategies can reduce product concentration relative to primer concentration and can reduce total product concentration in scaling up cPCR reaction to a high level of multiplexing, as illustrated in the figure (85% and 86% reduction are achieved based on simulation).

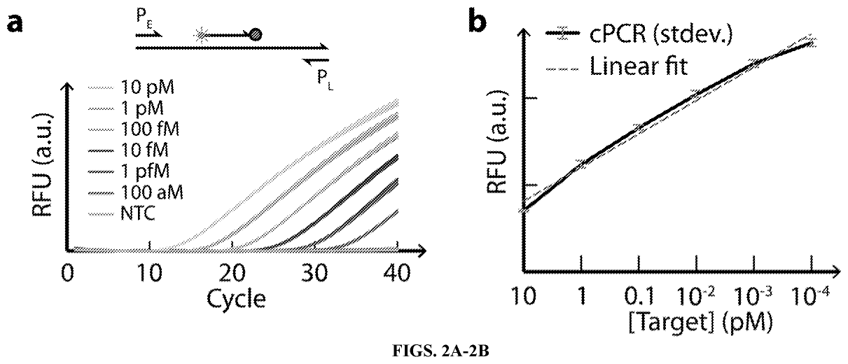

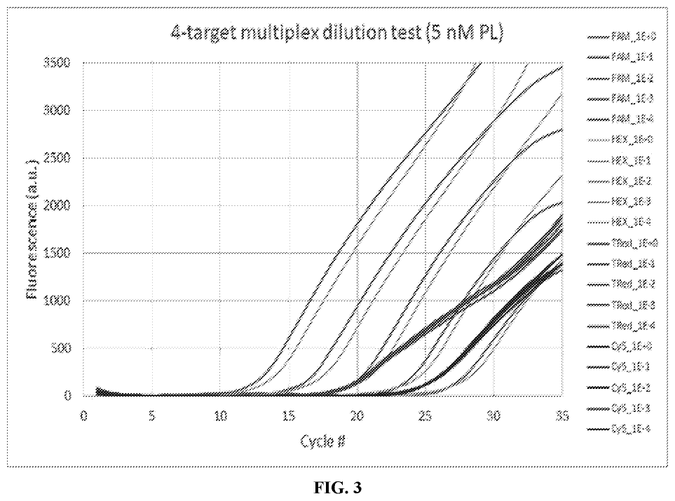

FIG. 2A depicts qPCR amplification, where traces confirm the two-phase reaction exhibited by cPCR. FIG. 2B demonstrates where cPCR shows a large log-linear dynamic range (5-log).

### Example 2—Four-Target Multiplex cPCR Read Out Via Real-Time PCR

Following a typical workflow for compression PCR (FIG. 1A), multiplexed cPCR is tested by mixing four sets of target templates and primer strands in the same reaction, with each set previously validated in individual cPCR tests. The amplicon, primers and probe sequences of each target are shown in Table 1.

TABLE 1
Sequences used in a 4-plex cPCR test.

| Target | Sequence | | SEQ ID |
|---|---|---|---|
| No. | Type | Sequence | No. |
| Target 1 | Amplicon | GACCCCAAAATCAGCGAAATGCACCCCG | 1 |
| | | CATTACGTTTGGTGGACCCTCAGATTCAA | |
| | | CTGGCAGTAACCAGA | |
| | P_E | GACCCCAAAATCAGCGAAAT | 2 |
| | P_L | TCTGGTTACTGCCAGTTGAATCTG | 3 |
| | Probe | ACCCCGCATTACGTTTGGTGGACC | 4 |
| Target 2 | Amplicon | TCCTCAACTCACTCTTCGAGCGTCTCAAT | 5 |
| | | GAAGGACATTCAAAGCCAATTCGAGCAG | |
| | | CTGAAACTGCGGTGGGAGTCTTATCCCA | |
| | | ATTTGGTCAAGAGCACCG | |
| | P_E | TCCTCAACTCACTCTTCGAGCG | 6 |
| | P_L | CGGTGCTCTTGACCAAATTGG | 7 |
| | Probe | CCAATTCGAGCAGCTGAAACTGCGGTG | 8 |
| Target 3 | Amplicon | CTGCAGATTTGGATGATTTCTCCAAACAA | 9 |
| | | TTGCAACAATCCATGAGCAGTGCTGACTC | |
| | | AACTCAGGCCTAAACTCATGCAGACCAC | |
| | | ACAAGG | |
| | P_E | CTGCAGATTTGGATGATTTCTCC | 10 |
| | P_L | CCTTGTGTGGTCTGCATGAGTTTAG | 11 |
| | Probe | ATTGCAACAATCCATGAGCAGTGCTGAC | 12 |
| | | TC | |
| Target 4 | Amplicon | AGATTTGGACCTGCGAGCGGGTTCTGAC | 13 |
| | | CTGAAGGCTCTGCGCGGACTTGTGGAGA | |
| | | CAGCCGCTC | |
| | P_E | AGATTTGGACCTGCGAGCG | 14 |
| | P_L | GAGCGGCTGTCTCCACAAGT | 15 |
| | Probe | TTCTGACCTGAAGGCTCTGCGCG | 16 |
| Control | Amplicon | TTACAAACATTGGCCGCAAATTGCACAA | 17 |
| | | TTTGCCCCCAGCGCTTCAGCGTTCTTCGG | |
| | | AATGTCGCGC | |
| | P_E | TTACAAACATTGGCCGCAAA | 18 |
| | P_L | GCGCGACATTCCGAAGAA | 19 |
| | Probe | ACAATTTGCCCCCAGCGCTTCAG | 20 |

In this experiment, all four pairs of primers are included at 5 nM for each limiting primer, and 250 nM for each excess primer. Taqman probes for real-time detection are also included at 250 nM each. Template amplicons are added in the range from 10 aM to 1 pM. To test the dynamic range, sequence crosstalk and signal linearity of multiplexed cPCR, the concentrations of two of the four targets are varied from 1 pM down to 10 aM in a tenfold dilution series, and kept the concentration of the other two constant at 10 fM. cPCR reaction is run with a 60-cycle thermal cycling program, and the annealing step is set to 55 °C and 12.5 min per cycle.

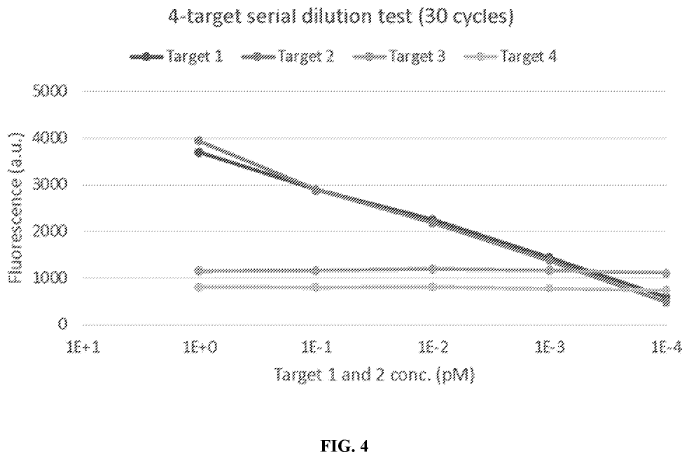

The real-time amplification performance is monitored using four-channel qPCR readout (one channel per target) on a RT-PCR instrument (Bio-Rad CFX Opus). Due to fluorescence crosstalk between different channels, signals are corrected for background with manually adjusted cycle limits. After background subtraction, expected two-phase amplification signals are expected for all samples in all channels, and all channels show exponential-to-linear transition at similar signal levels (FIG. 3). For the two targets with varying concentration, parallel and equally spaced amplification signals are observed, as expected for dilution series samples, suggesting linear amplification of each signal independent of other targets present. For the two fixed targets, overlapping signals are observed as expected. Their signal intensity is extracted at cycle 30, and log-linearly decreased signal intensity is observed as a function of input target concentration (FIG. 4).

During later stage of the amplification, higher signals are observed, likely due to non-specific primer binding. However, amplification results from such non-specific binding are expected to have distinct sequence from on-target amplicons.

### Example 3—Four-Target Multiplex cPCR Read Out Via Next-Generation Sequencing

The end-point cPCR performance is also evaluated by combining different samples from Example 2 and pooled next-generation sequencing. After the cPCR reactions, samples are first spiked in with a double-stranded control amplicon, and converted to double-stranded DNA by a single cycle of PCR, where all four limiting primers are included. Samples are then barcoded and pooled using NEB's indexed primer sets (NEBNext Multiplex Oligos for Illumina) and following protocols slightly adapted from the manufacturer's recommendation. Briefly, the converted double-stranded DNA samples are first purified with AmPure XP magnetic beads. Next, purified samples are ligated with NEB universal adaptor and treated with USER enzyme following end prep, and purified with magnetic beads again. Then, library PCR is performed with NEB indexed primers and purified with magnetic beads a third time. Finally, barcoded samples are normalized on Qubit and mixed for high-throughput sequencing on a MiSeq machine. After sequencing, reads are locally aligned with bowtie2 to a library that includes all target amplicon sequences, and full-length sequence matches with a low sequencing error (edit distance<=2) are selected and counted. Read counts are then normalized against the control amplicon, as well as with a per-target normalization factor to account for target-specific amplification biases.

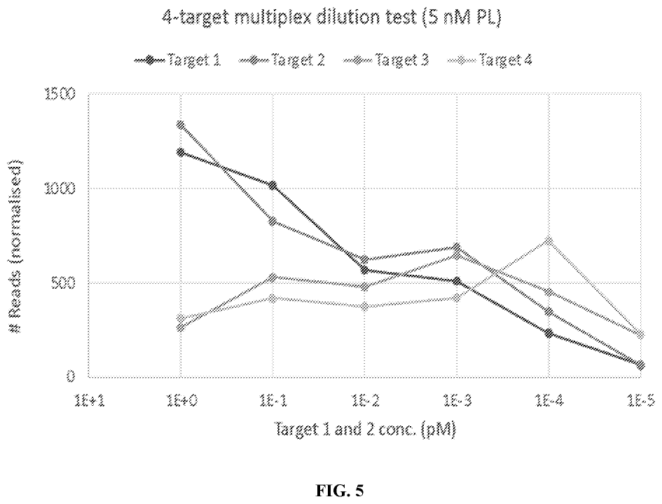

Final normalized sequencing reads show a linear relationship against input target concentration, as expected (FIG. 5).

### Example 4—Four-Target Multiplex cPCR with Updated Conditions and Wider Dynamic Ranges

The same 4-plex cPCR templates and primers from Examples 2 and 3 are reused and repeated with improved testing conditions. In particular, higher limiting primer concentration (25 nM or 10 nM), together with higher excess primer concentration (1.25 μM or 500 nM) are used to maintain excess-to-limiting primer concentration ratio. Shorter RT-PCR reactions (40 cycles) with shorter extension time per cycle (5 min) are preformed. A wider dynamic range of original target concentration is also tested. In particular, the concentrations of the first two targets are varied from 100 pM to 10 aM (e.g. 7 logs) in a tenfold dilution series, while the other two are kept at a constant 100 fM.

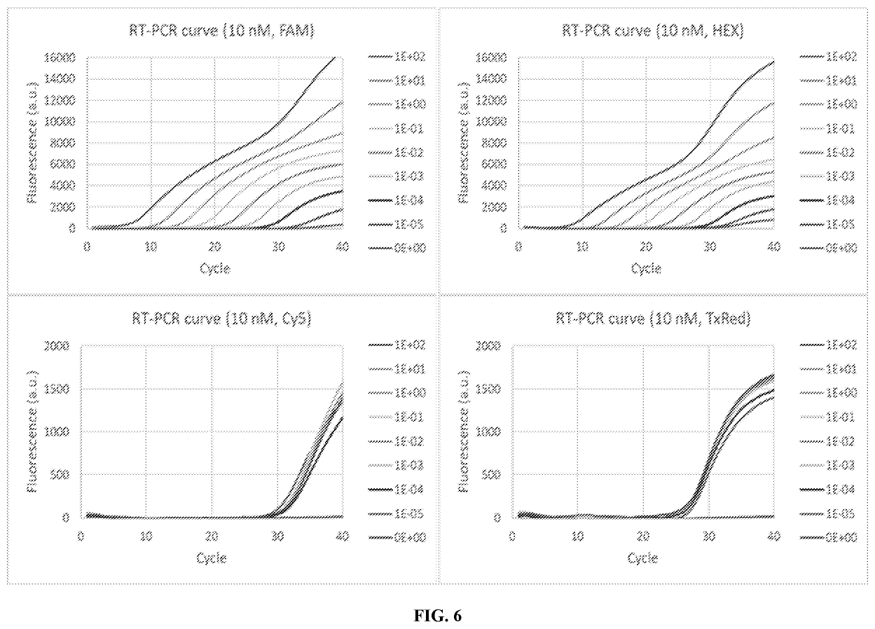

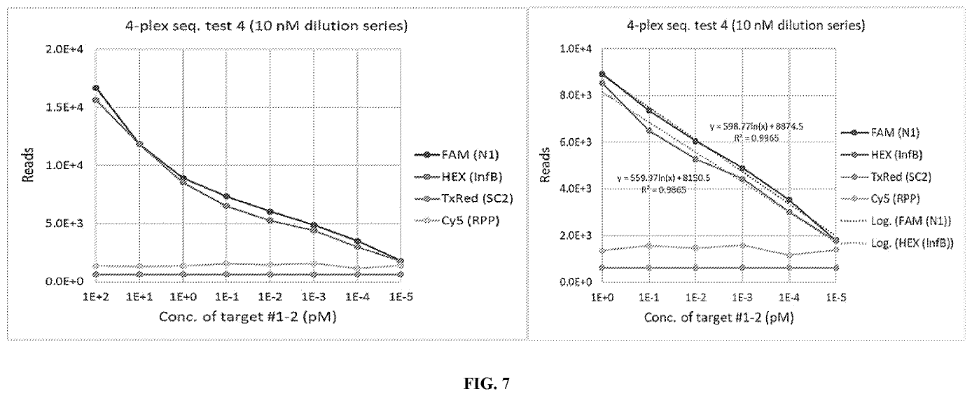

From real-time fluorescence traces on the RT-PCR, two clear stages of amplification (exponential and linear) of the cPCR reaction are observed (FIG. 6). No manual background subtraction is performed. A second phase of signal increase is also observed, likely due to non-specific primer binding, that starts at cycle 25-35, depending on the original target concentration (FIG. 6). In the range from 1 pM to 10 aM, good linearity between end-point fluorescence signal level and log (original target concentration) is observed, with R²=0.997 and 0.987 for the two targets, respectively (FIG. 7).

The above reaction products are then converted from ssDNA to dsDNA. After spiking in two double-stranded control amplicons, a library is prepared for sequencing following similar procedure as in Example 3. Briefly, samples are ligated with NEBNext universal adaptor, barcoded with NEBNext Multiplex Index Oligos, and pooled before sequencing on an MiSeq instrument. After sequencing, reads are analyzed using the same procedure as in Example 3. Briefly, raw reads are locally aligned with bowtie2, filtered by full-length sequence match and edit distance threshold (<=2), normalized by the control amplicons and manually scaled by a per-target normalization factor.

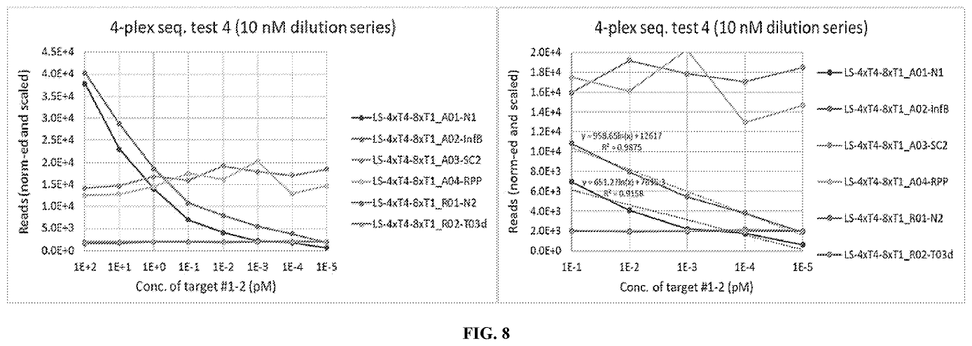

Final normalized sequencing reads show a monotonic decreasing relationship against input target concentration, and linear relationship in the low-concentration range from 100 fM to 10 aM (FIG. 8).

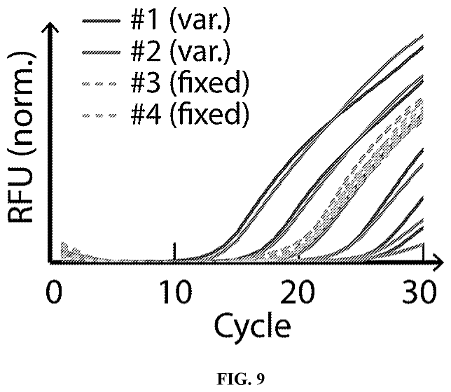

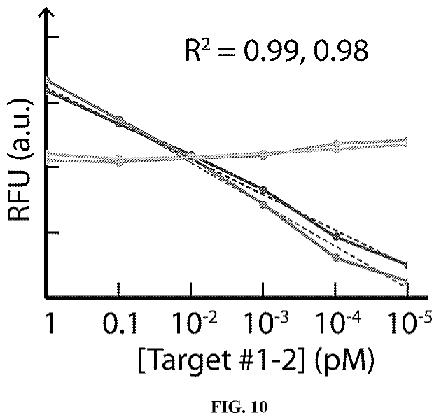

A 4-target multiplexed cPCR was conducted with varying concentrations of the target for #1 and #2 (in FIG. 9), and fixed concentrations for #3 and #4 (in FIG. 9), and the qPCR amplification traces are shown in FIG. 9. The endpoint signal has high linearity, as depicted in FIG. 10.

### Example 5—Eight-Target Multiplex cPCR

An eight-target multiplex cPCR is performed using custom designed primers against human genome targets. In particular, 96 SNPs in the human genome having significant variant allele frequency are selected. A custom primer pool design algorithm is used to minimize crosstalk between any pairs of primers, while maintaining a tight binding energy distribution. Table 2 shows the sequences of the targets and primers used. Synthetic DNA (IDT ultramers) is used as templates for the test. For the cPCR reaction, limiting primers are used at 10 nM each, and excess primers are used at 500 nM. Forty cycles of PCR thermocycling are performed with 5 min annealing and extension per cycle at 55 °C. Out of the 8 targets, the concentrations of the first four targets are varied from 10 pM to 10 aM (e.g. 6 logs) in a tenfold dilution series, and the other two targets are kept at a constant 10 fM. After the first PCR reaction, the reaction products are converted from ssDNA to dsDNA, after spiking in two double-stranded control amplicons. NGS sequencing libraries are prepared and data analysis follow the same procedure as for the 4-plex cPCR experiment in Example 4.

TABLE 2
Sequences used in a 8-plex cPCR test.

| Target | Molecule | | SEQ ID |
|---|---|---|---|
| No. | Type | Sequence | No. |
| Target | Template 1 | GATCTGCTGTTTCACTTTGGGTTAAAATCTTT | 21 |
| #1 | (T01) | TGCTTTCATATTGAGCTTAAGAGTTCAGAAC | |
| | | ACTGATGGAAAAAACTTGGCTCTTTGTTATT | |
| | | TTTCAGAAATGTATTTTT | |
| | Limiting | ATTTCTGAAAAATAACAAAGAGCCAAGT | 22 |
| | Primer 1 | | |
| | (PL01) | | |
| | Excess | TGCTGTTTCACTTTGGGTTAAAA | 23 |
| | Primer | | |
| | (PE01) | | |
| Target | T02 | GCCATTATTTGTATCATGGGTAGCTTGATCC | 24 |
| #2 | | TCAGAGCACCTTGCAGGCTGCTTTCTAGAGT | |
| | | AAGGATTAGAAGATTAGAAATATGTGAACA | |
| | | AACTCCCTCCTCTACACGCTTTGA | |
| | PL02 | TCAAAGCGTGTAGAGGAGG | 25 |
| | PE02 | GCCATTATTTGTATCATGGGTAGC | 26 |
| Target | T03 | TTGGCTCCCAACAGTGAACTCAGCTACATGC | 27 |
| #3 | | ATTCATTTAGAGAATAAAAGTTAATCCGGGT | |
| | | TTGGAATCCATTGCATTTGTTTTGGGCACAC | |
| | | TGGTGTCT | |
| | PL03 | ACCAGTGTGCCCAAAACAAA | 28 |
| | PE03 | TTGGCTCCCAACAGTGAAC | 29 |
| Target | T04 | CCACGCTGAATACTTCTTTCTAATACACATT | 30 |
| #4 | | GAATCGACTACCAATATGGTGTTTTGGAAAA | |
| | | GCTTACCACTCCAATTTCATCACAAGTCTGT | |
| | | ATAGACCACATTACTTACAAAA | |
| | PL04 | CACGCTGAATACTTCTTTCTAATACACA | 31 |
| | PE04 | TTTTGTAAGTAATGTGGTCTATACAGAC | 32 |
| Target | T05 | CCCAAAGTGTTGGGATTACAGTCATGAGCC | 33 |
| #5 | | ACCACACCTGGCCCATCATAACTATTTTCGA | |
| | | GTTTGCATGGAACCCACTTTGCTCCCAGCTT | |
| | | TCTGCGCTGGTTGCCCTTAGGC | |
| | PL05 | GCAGAAAGCTGGGAGCAAAG | 34 |
| | PE05 | CCCAAAGTGTTGGGATTACAGT | 35 |
| Target | T06 | TGTAAATCTGTTCTTCTCAGTTGAAAGGATA | 36 |
| #6 | | AAAGCTAAATAGGAATTGCATGAAAGCTAA | |
| | | ATAGGATTTTTTTCTGTCATCGTTTATTATCA | |
| | | CAATTATCCTCCCCA | |
| | PL06 | TGGGGAGGATAATTGTGATAATAAACG | 37 |
| | PE06 | AGTTGAAAGGATAAAAGCTAAATAGGAA | 38 |
| Target | T11 | TGGCGCTTGCTAAAGAGCATTTAACTCATCC | 39 |
| #7 | | TGATTCACCGCAGGATTGGGCTGGCTTTCAA | |
| | | ACTCCTGACAGTGAATTACTGCTCTTGTAAT | |
| | | GACGAATCCA | |
| | PL11 | GGCGCTTGCTAAAGAGCA | 40 |
| | PE11 | TGGATTCGTCATTACAAGAGCAGT | 41 |
| Target | T12 | TAGCAATATTATTGTACATATCACAGGGTAT | 42 |
| #8 | | ACCTGTGTGAGAGTTTCTTTTGCGTATATAT | |
| | | CGAGATGGAAATTGCTCATAGAGTAGGTGA | |
| | | ATACTTGA | |
| | PL12 | TCAAGTATTCACCTACTCTATGAGCAAT | 43 |
| | PE12 | TAGCAATATTATTGTACATATCACAGGGT | 44 |

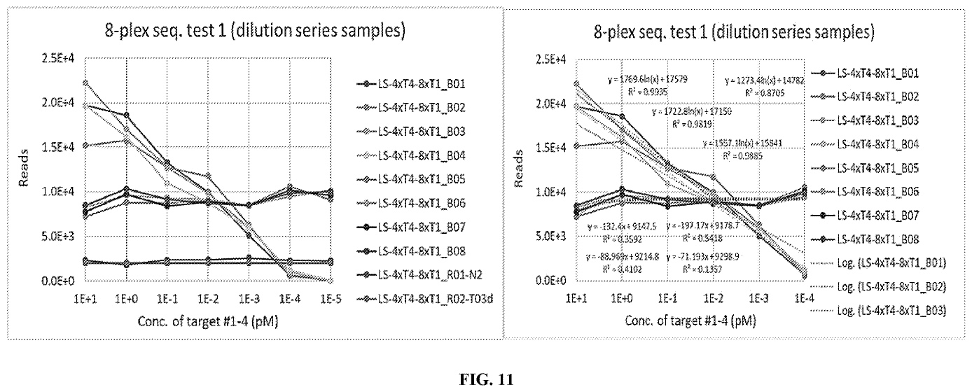

A clear linear decreasing relationship is observed between the normalized sequencing read counts and the logarithm of input target concentration, across the target concentration range from 10 pM down to 100 aM (i.e. 5 logs dynamic range) (FIG. 11). Linear fit shows an R² coefficients of 0.982, 0.871, 0.994, 0.989 for the four targets, respectively. The lowest concentration (10 aM) is not clearly detected, likely due to a lack of sensitivity of this test design. The other four targets with constant input concentration show a fixed sequencing readout.

### Example 6—Eight-Target Multiplex cPCR

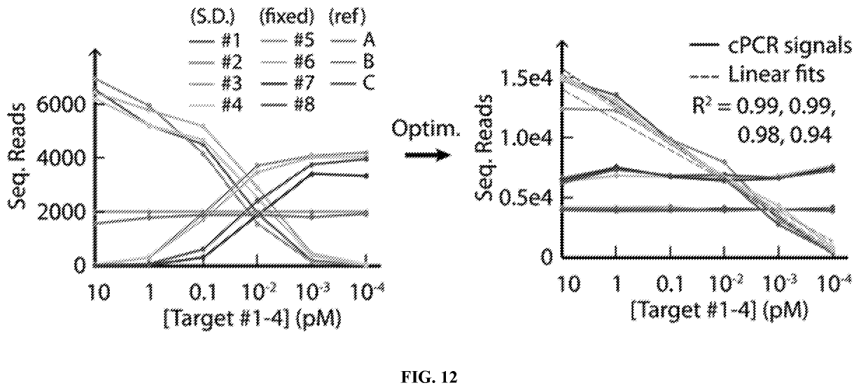

A separate eight-target multiplex experiment comparing different experimental conditions is performed. For the cPCR reaction, limiting primers are used at 10 nM each, and excess primers are used at 500 nM. Forty cycles of PCR thermocycling are performed with 5 min annealing and extension per cycle at 55 °C. Different enzyme amount (1:5 ratio) was used for comparison. FIG. 12 shows that, only with appropriately adjusted enzyme amount, cPCR reaction shows linear response against the log of input concentration, and constant signal for targets kept at fixed concentration.

### Example 7—96-Target Multiplex cPCR

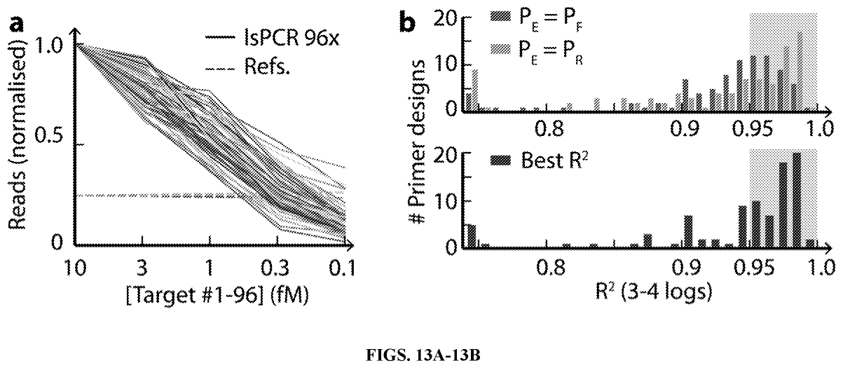

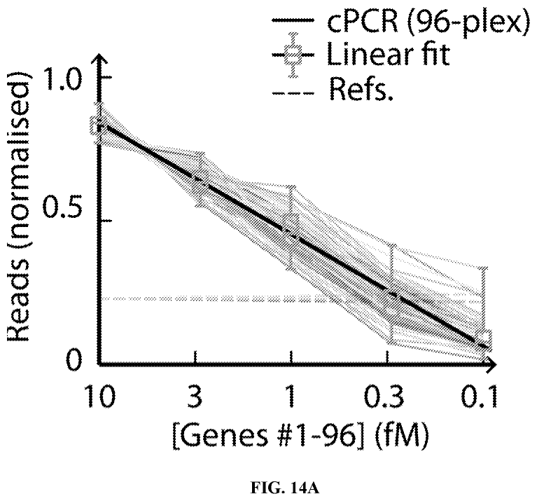

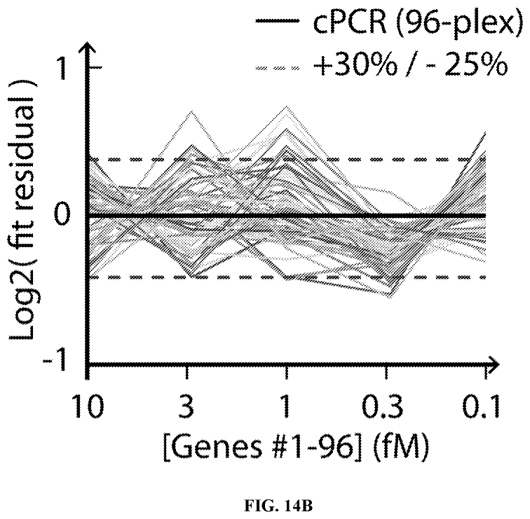

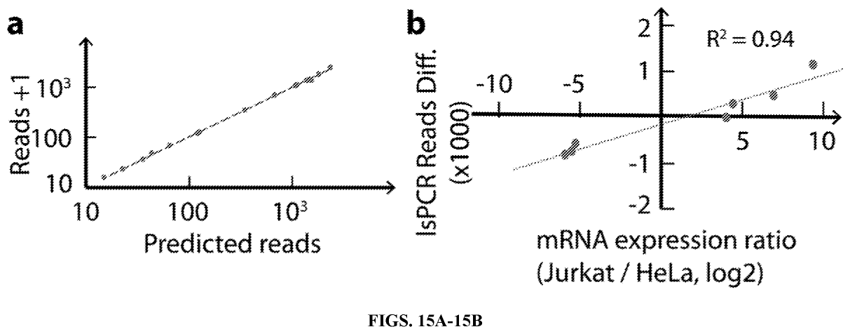

A 96-target multiplex cPCR is performed using custom designed primers against human genome SNP targets with significant variant allele frequency. A custom primer pool design algorithm is used to minimize crosstalk between any pairs of primers, while maintaining a tight binding energy distribution. The test is performed with human genomic DNA sample (Sigma), over a dilution series from 10 fM to 0.1 fM. For the cPCR reaction, limiting primers are used at 5 nM each, and excess primers are used at 150 nM. Forty cycles of PCR thermocycling are performed with 12.5 min annealing and extension per cycle at 55 °C. After the first PCR reaction, the reaction products are converted from ssDNA to dsDNA, after spiking in two double-stranded control amplicons. NGS sequencing libraries are prepared and data analysis follow the same procedure as in Example 4. FIGS. 13A-13B show that, 76% (73 out of 96) targets showed a linear response (R²>0.95) against log of input gDNA concentration. FIG. 14A shows cPCR testing on a serial dilution sample with high linearity (R²>0.95 for 75% of primer pairs. FIG. 14B shows accurate detection (+/−25%) on part with RNA-seq.

### Example 8—30-Target Multiplex cPCR on Total RNA Sample

An eight-target multiplex cPCR is performed using custom designed primers against human transcriptome targets. In particular, 30 human mRNA targets were designed in such a way that span the full dynamic range of gene expression, and also show significant differential gene expression between HeLa and Jurkat cell lines, based on a previously reported RNA-seq dataset. HeLa and Jurkat total RNA samples (BioChain) were reverse transcribed with poly-dT primer, and purified with magnetic beads before cPCR reaction. For the cPCR reaction, limiting primers are used at 5 nM each, and excess primers are used at 250 nM. Forty cycles of PCR thermocycling are performed with 12.5 min annealing and extension per cycle at 55 °C. cDNA samples were diluted to an effective abundance of about 100 cells. After the first PCR reaction, the reaction products are converted from ssDNA to dsDNA, after spiking in two double-stranded control amplicons. NGS sequencing libraries are prepared and data analysis follow the same procedure as in Example 4.

FIG. 15A shows that, after cPCR and high-throughput sequencing, genes spanning the full 4 logs of gene expression (from the reported RNA-seq dataset) were detected, although not all targets are detected, which could be due to sample-to-sample variation. The data show good agreement with expected gene abundances, after linear fitting and correcting for sequence-specific amplification bias. FIG. 15B further shows a linear correlation (R2=0.94) of differential gene expression compared to previous RNA-seq reports.

### Example 9—Single-Cell Multiplexed cPCR Sequencing with a hPBMC Sample

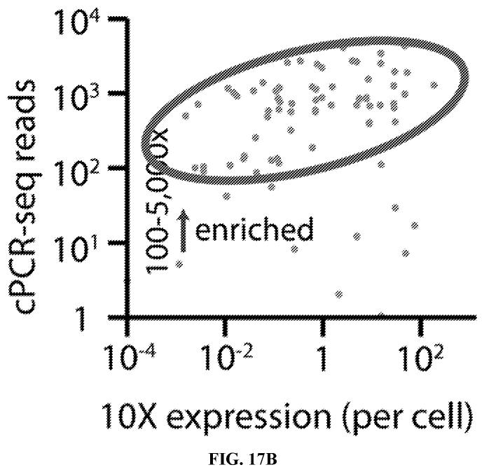

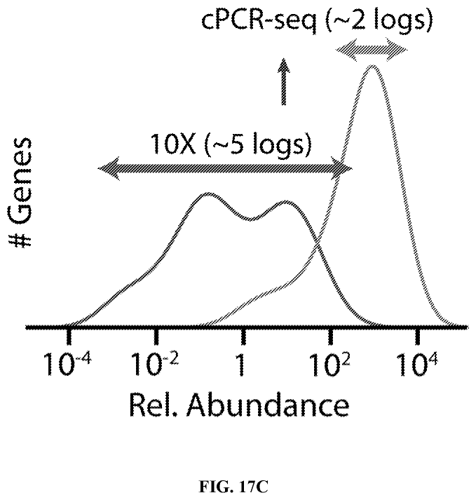

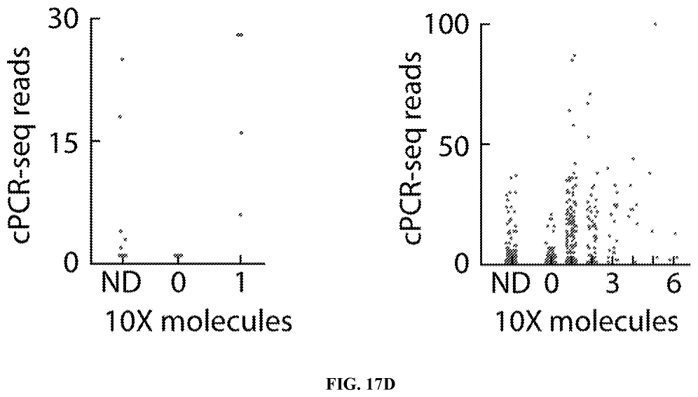

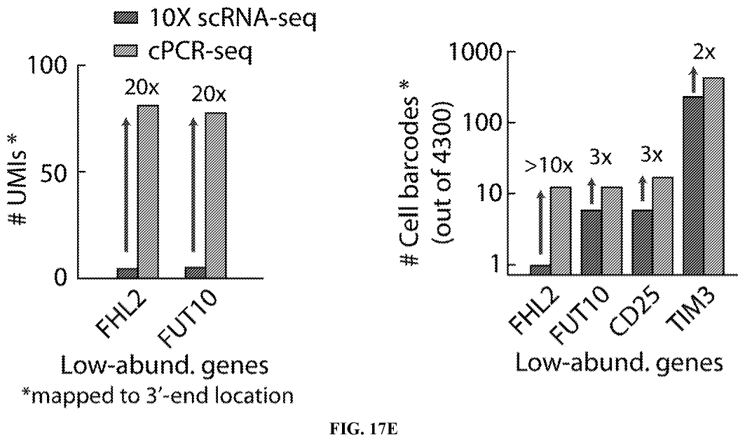

A single-cell multiplexed cPCR sequencing test is performed on human peripheral blood mononuclear cell (hPBMC, Lonza) samples, on a panel of 60 gene targets. hPBMC samples were prepared following standard 10× Genomics 3′ mRNA profiling workflow (MD Anderson Advanced Technology Genomics Core), and analyzed using cellranger pipeline (10×). Following sequencing library preparation using 10× Genomics workflow, cPCR was performed using custom designed primers against 121 gene targets. These targets were chosen from 10× sequencing results, to span a dynamic range of 3×10⁴ (or greater, but limited by sequencing depth). A PCR test showed 60 of the designed primers generated well-amplified sequences that aligned well to human genome reference database at expected loci. Multiplexed cPCR reaction was performed with 2 nM limiting primers, and a common primer overlapping with the Illumina Read 1 primer as the excess primer for all targets (7.2 μM) (see FIG. 1E). Sample preparation and high-throughput sequencing was performed in a similar fashion to Example 8. There were high-, medium- and low-abundance groups to span >4 logs of dynamic range as reported on a 10× single-cell profiling test (FIG. 17A).

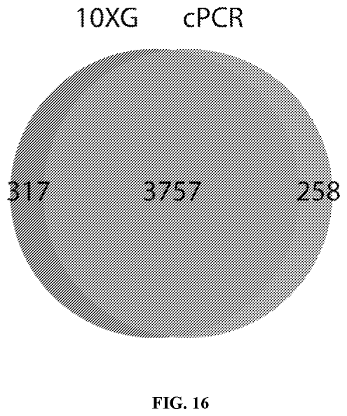

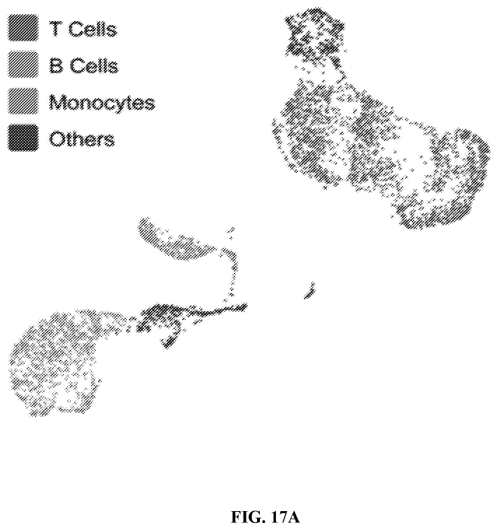

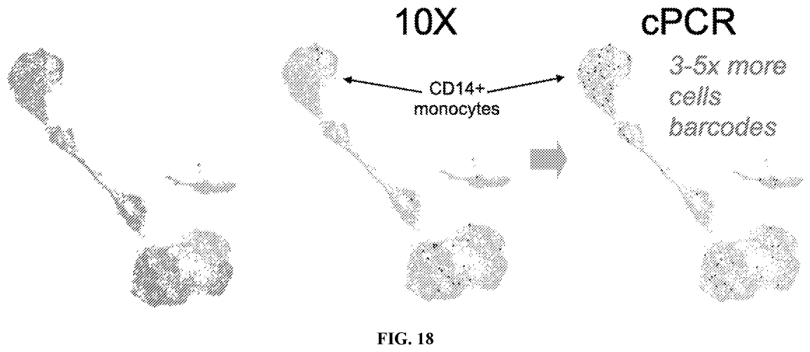

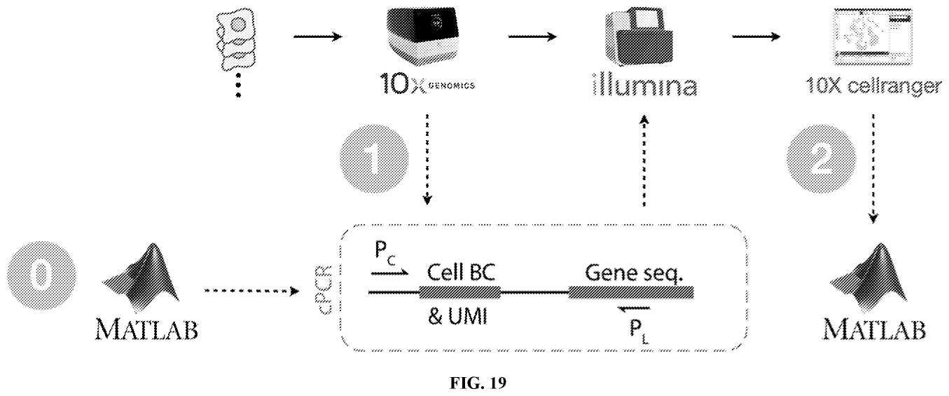

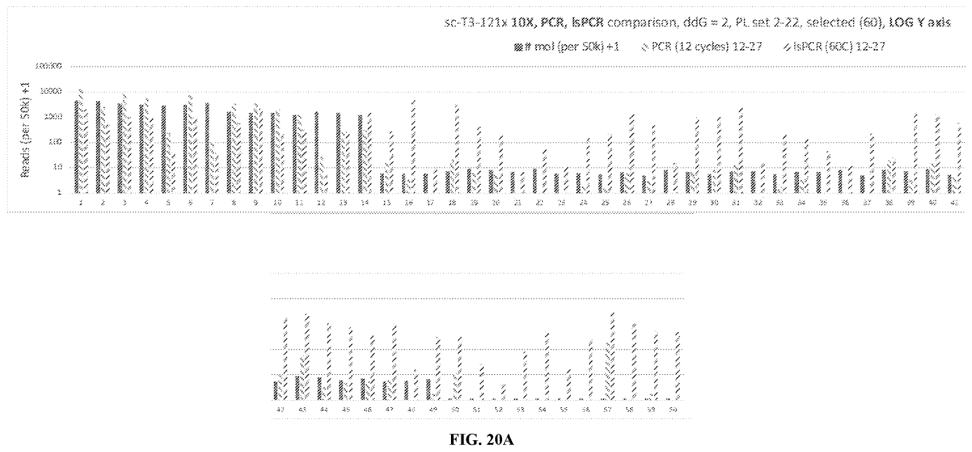

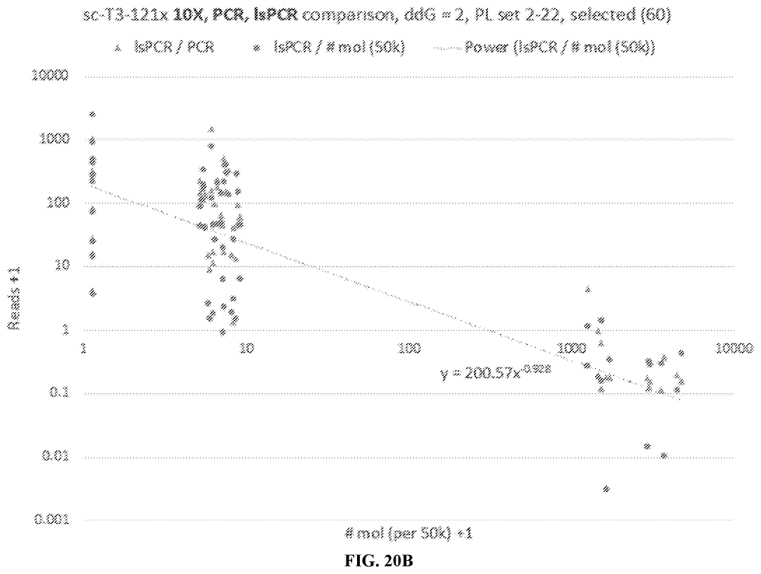

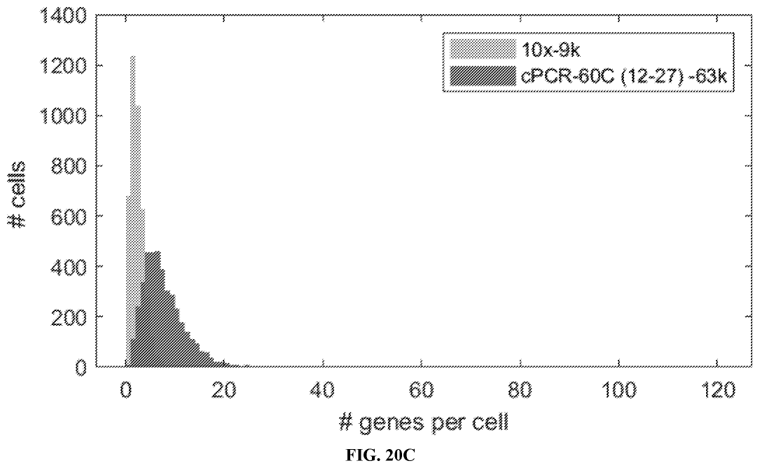

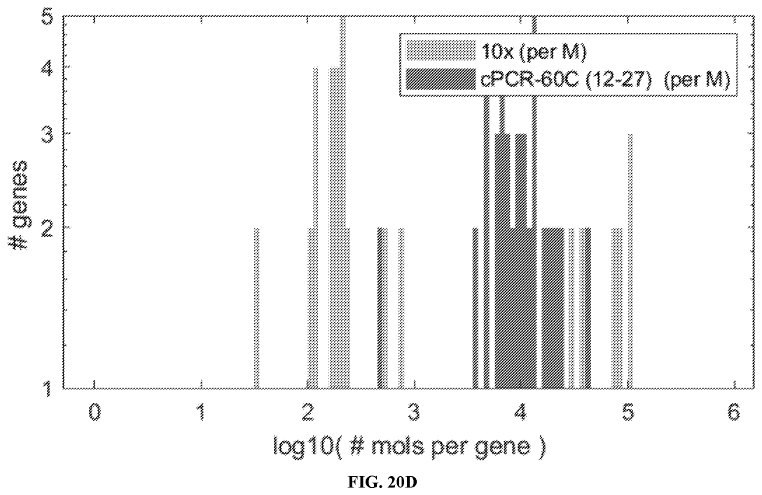

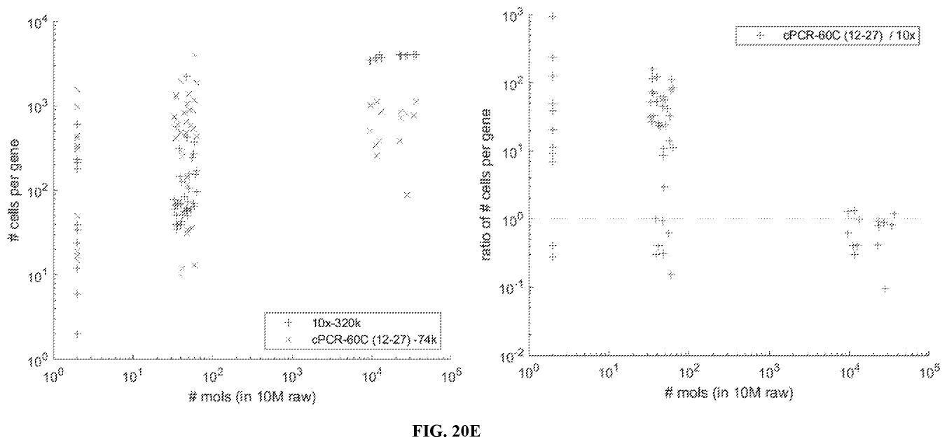

Bulk sequencing analysis (i.e., without cell barcode and UMI) showed that, within the set of 60 well-designed targets, cPCR showed close-to-uniform target read depth across the entire dynamic range of genes tested (3×10⁴), compared with 10× Genomics 3′ mRNA assay, and the low abundance genes were 100-5,000× enriched (FIGS. 17B, and 17C), achieving a ~1,000× reduction in overall dynamic range. When normalized to the same total number of sequencing reads (50,000), cPCR allocated ~10× fewer sequencing reads per gene for the most abundant gene group, 10~100× more reads for the median-to-low abundance genes, and up to ~1000× more reads for the lowest abundance genes tested (FIGS. 20A and 20B). After cell barcode demultiplexing and UMI analysis, cPCR (74,000 total reads) detected 26897 distinct cell barcodes, with an overlap of 4058 out of the 4074 total cell barcodes detected in a 10× single-cell dataset (with 10,000,000 randomly sub-sampled reads), representing a >99% barcode coverage. Specifically, 4015 of detected barcodes showed at least 5 distinct reads, out of which 3757 were common with 10× dataset (92% coverage, FIG. 16). In the mutually detected single cells, particularly for the low-abundance genes, cPCR detected significantly more mapped reads (mapped to same 3′end mRNA location) compared to 10× sequencing (FIG. 17D). Within the 4058 mutually detected single cells and out of the 46 medium-to-low abundance gene targets, cPCR detected up to >20 genes in single cells (mean 7.21, std 4.13), which is roughly 4× higher compared to <=5 genes for standard 10× analysis (mean 1.83, std 1.45, FIG. 20C). Similarly, in the mutually detected single cells, particularly for the low-abundance genes, the map reads covered up to 20× more molecular UMIs and 2-10× more cell barcodes expressing this gene (FIG. 17E). For each medium-to-low abundance gene target, cPCR detected 10-100× more reads (FIG. 20D) as well as 10-100× more single cells expressing the target gene (FIG. 20E), as compared to the 10× dataset. These results suggest that cPCR allows >100-fold effective sequencing depth as compared to the standard 10× single-cell 3′ mRNA analysis, allowing for much deeper (more molecules, more cells expressing target genes) molecular profiling of the transcriptome with single-cell resolution. Additionally, cPCR has a 3-5× higher barcode coverage relative standard 10× sequencing dataset for multiplexed single-cell sequencing (FIG. 18). FIG. 19 depicts an exemplary design and experiment workflow for single-cell sequencing with cPCR.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method for sequencing a plurality of target sequences in a nucleic acid mixture sample, the method comprising:
a. subjecting the nucleic acid mixture sample to an asymmetric PCR reaction (i) in a multiplexed format for the plurality of target sequences, and (ii) for each individual target sequence, using an excess primer and a limiting primer each comprising at least a sequence portion capable of binding to a separate strand of the individual target sequence, wherein the excess primer is at a concentration at least 5 fold higher than the limiting primer;
b. preparing a high-throughput sequencing or hybridization library from nucleic acid products amplified from the asymmetric PCR reaction.

2. The method of claim 1, wherein the excess primer is shared among all or some of the target sequences and is capable of binding to a common primer binding site shared among all or some of the target sequences.

3. The method of claim 2, wherein the concentration of the excess primer being at least 5 fold higher is determined based on per target sequence basis.

4. A method for sequencing a plurality of target sequences in a nucleic acid mixture sample, the method comprising:
a. subjecting the nucleic acid mixture sample to an asymmetric PCR reaction in a multiplexed format for the plurality of target sequences, wherein the asymmetric PCR reaction involves three primers for each target sequences: (i) a forward primer, (ii) a reverse primer, and (iii) an excess primer; wherein both the forward and reverse primers are limiting primers, while the excess primer is shared among all or some of the target sequences and is capable of binding to amplicons generated by the forward and reverse primers;
b. preparing a high-throughput sequencing or hybridization library from nucleic acid products amplified from the asymmetric PCR reaction.

5. The method of any of claims 1 to 4, further comprising: converting single-stranded nucleic acids produced from the asymmetric PCR reaction into double-stranded nucleic acids.

6. The method of any of claims 1 to 5, wherein the excess primer is at a concentration at least 10 fold higher than the limiting primer or limiting primers, resulting in preferential amplification of the strand targeted by the excess primer.

7. The method of any of claims 1 to 6, further comprising: detecting the nucleic acid products amplified from the asymmetric PCR reaction using a sequencing method, a hybridization method, a microarray method, or a quantitative PCR method.

8. The method of any of claims 1 to 7, wherein the preparation of the high-throughput sequencing or hybridization library comprises a ligation reaction.

9. The method of any of claims 1 to 7, wherein the preparation of the high-throughput sequencing or hybridization library comprises a polymerase-based reaction.

10. The method of any of claims 1 to 9, further comprising:
c. obtaining sequencing reads data from the high-throughput sequencing library, wherein the sequencing reads data (i) reflect a transformed abundance of the plurality of target sequences in the nucleic acid mixture sample and (ii) exhibit a compressed dynamic range relative to the nucleic acid mixture sample, and
d. determining the original abundance of the plurality of target sequences in the nucleic acid mixture sample based on the transformed abundance.

11. The method of any of claims 1 to 9, further comprising:
c. obtaining abundance readout from the high-throughput hybridization library, wherein the abundance readout (i) reflects a transformed abundance of the plurality of target sequences in the nucleic acid mixture sample and (ii) exhibits a compressed dynamic range relative to the nucleic acid mixture sample, and
d. determining the original abundance of the plurality of target sequences in the nucleic acid mixture sample based on the transformed abundance.

12. The method of any of claims 1 to 11, wherein the plurality of target sequences in the nucleic acid mixture sample comprise an original dynamic range of at least 50:1, 100:1, 1000:1, 10000:1, or 100000:1.

13. The method of any of claims 1 to 11, wherein the plurality of target sequences in the nucleic acid mixture sample comprise an original dynamic range of between 50:1 and 100:1, between 50:1 and 1000:1, between 50:1 and 10000:1, between 100:1 and 500:1, between 500:1 and 1000:1, between 500:1 and 5000:1, between 500:1 and 10000:1, between 1000:1 and 5000:1, or between 1000:1 and 10000:1.

14. The method of any of claims 11 to 13, wherein the original abundance is log-transformed into the transformed abundance with an optional linearity correction.

15. The method of any of claims 11 to 13, wherein the original abundance is determined based on (i) the transformed abundance and (ii) the concentration of the limiting primer used in the asymmetric PCR reaction.

16. The method of any of claims 11 to 13, wherein the compressed dynamic range is at or below 2:1, 3:1, 4:1, 5:1, 6:1, 7:1, 8:1, 10:1, 25:1, 50:1, 100:1, or 250:1.

17. The method of any of claims 11 to 13, wherein the compressed dynamic range is between 2:1 and 3:1, between 2:1 and 4:1, between 2:1 and 5:1, between 5:1 and 10:1, between 5:1 and 15:1, between 5:1 and 20:1, between 10:1 and 50:1, between 10:1 and 100:1, between 50:1 and 100:1, between 50:1 and 250:1, or between 100:1 and 250:1.

18. The method of any of claims 1 to 17, wherein the multiplexed format is at least 10-plex, 20-plex, 50-plex, 100-plex, 200-plex, 300-plex, 400-plex, 500-plex, 750-plex, 1000-plex, 2000-plex, 5000-plex, 10000-plex, 20000-plex, 50000-plex, or 100000-plex.

19. The method of any of claims 1 to 17, wherein the multiplexed format is between 500-plex and 1000-plex, between 500-plex and 2000-plex, between 500-plex and 3000-plex, between 500-plex and 4000-plex, between 500-plex and 5000-plex, between 500-plex and 10000-plex, between 1000-plex and 2000-plex, between 2000-plex and 4000-plex, between 2000-plex and 6000-plex, between 2000-plex and 8000-plex, between 5000-plex and 8000-plex, between 5000-plex and 10000-plex, between 5000-plex and 20000-plex, between 5000-plex and 30000-plex, between 5000-plex and 40000-plex, between 5000-plex and 50000-plex, between 5000-plex and 60000-plex, between 15000-plex and 20000-plex, between 15000-plex and 30000-plex, between 15000-plex and 40000-plex, between 7000-plex and 100000-plex, between 10000-plex and 100000-plex, between 15000-plex and 100000-plex, between 25000-plex and 100000-plex, between 35000-plex and 100000-plex, between 45000-plex and 100000-plex, between 55000-plex and 100000-plex, between 65000-plex and 100000-plex, between 75000-plex and 100000-plex, or between 85000-plex and 100000-plex.

20. The method of any of claims 1 to 19, wherein the excess primer is at a concentration at least 2, 3, 4, 5, 6, 7, 8, 9, 10, 11, 15, 20, 25, 30, 35, 40, 45, 50, 75, 100, 150, 200 or 500 fold higher than the limiting primer.

21. The method of any of claims 1 to 19, wherein the excess primer is at a concentration between 2 and 5, between 2 and 10, between 5 and 10, between 5 and 15, between 10 and 16, between 10 and 20, between 15 and 30, between 20 and 30, between 25 and 50, between 30 and 70, between 40 and 60, between 40 and 70, between 40 and 80, between 50 and 100, between 50 and 150, between 70 and 200, or between 100 and 200 fold higher than the limiting primer.

22. The method of any of claims 1 to 19, wherein the excess primer is at a concentration about 20 to 50 fold higher than the limiting primer.

23. The method of any of claims 1 to 19, wherein the excess primer is at a concentration (i) about 50, 100, 150, 200, 250, 300, 350, 400, 450, 500, 600, 700, 800, 900 or 1000 nM, or (ii) about 2, 3, 4, 5, 6, 7, 8, 9, 10, 11, 12, 13, 14, 15, 16, 17, 18, 19, 20, 25, 30, 35, 40, 45, or 50 μM.

24. The method of any of claims 1 to 19, wherein the excess primer is at a concentration (i) at least 50, 100, 150, 200, 250, 300, 350, 400, 450, 500, 600, 700, 800, 900 or 1000 nM, or (ii) at least 2, 3, 4, 5, 6, 7, 8, 9, 10, 11, 12, 13, 14, 15, 16, 17, 18, 19, 20, 25, 30, 35, 40, 45, or 50 μM.

25. The method of any of claims 1 to 19, wherein the excess primer is at a concentration (i) between 50 and 1000, between 50 and 900, between 50 and 800, between 50 and 700, between 50 and 600, between 50 and 500, between 50 and 400, between 50 and 300, between 50 and 200, between 50 and 100, between 100 and 1000, between 150 and 1000, between 250 and 1000, between 350 and 1000, between 450 and 1000, between 550 and 1000, between 650 and 1000, between 750 and 1000, between 850 and 1000, between 100 and 900, between 150 and 800, between 250 and 700, between 350 and 600, between 450 and 500, between 150 and 250, between 250 and 350, between 350 and 450, between 450 and 550, between 550 and 650, between 650 and 750, or between 750 and 850 nM; or (ii) between 2 and 50, between 3 and 45, between 4 and 40, between 5 and 35, between 6 and 30, between 7 and 25, between 8 and 20, between 9 and 15, between 3 and 50, between 4 and 50, between 5 and 50, between 6 and 50, between 7 and 50, between 8 and 50, between 9 and 50, between 10 and 50, between 15 and 50, between 20 and 50, between 25 and 50, between 30 and 50, between 35 and 50, between 40 and 50, between 45 and 50, between 2 and 45, between 2 and 40, between 2 and 35, between 2 and 30, between 2 and 25, between 2 and 20, between 2 and 15, between 2 and 10, and between 2 and 5 μM.

26. The method of any of claims 1 to 19, wherein the limiting primer is at a concentration about 0.01, 0.02, 0.05, 0.1, 0.2, 0.3, 0.4, 0.5, 0.75, 1, 1.25, 1.5, 1.75, 2, 2.5, 3, 4, 5, 6, 7, 8, 9, 10, 12.5, 15, 20, 30, 40 or 50 nM.

27. The method of any of claims 1 to 19, wherein the limiting primer is at a concentration at least 0.01, 0.02, 0.05, 0.1, 0.2, 0.3, 0.4, 0.5, 0.75, 1, 1.25, 1.5, 1.75, 2, 2.5, 3, 4, 5, 6, 7, 8, 9, 10, 12.5, 15, 20, 30, 40 or 50 nM.

28. The method of any of claims 1 to 19, wherein the limiting primer is at a concentration (i) between 2.5 and 25, between 5 and 20, between 7.5 and 17.5, between 10 and 15, between 5 and 100, between 5 and 90, between 5 and 80, between 5 and 70, between 5 and 60, between 5 and 50, between 5 and 40, between 5 and 30, between 5 and 20, between 5 and 10, between 10 and 100, between 15 and 100, between 25 and 100, between 35 and 100, between 45 and 100, between 55 and 100, between 65 and 100, between 75 and 100, between 85 and 100, between 10 and 90, between 15 and 80, between 25 and 70, between 35 and 60, between 45 and 50, between 15 and 25, between 25 and 35, between 35 and 45, between 45 and 55, between 55 and 65, between 65 and 75, or between 75 and 85 nM; or (ii) between 0.01 and 2, between 0.02 and 2, between 0.05 and 2, between 0.1 and 2, between 0.2 and 2, between 0.3 and 2, between 0.4 and 2, between 0.5 and 2, between 0.75 and 2, between 1 and 2, between 1.25 and 2, between 1.5 and 2, between 1.75 and 2, between 0.02 and 1.75, between 0.05 and 1.5, between 0.1 and 1.25, between 0.2 and 1, between 0.3 and 0.75, between 0.4 and 0.5, between 0.02 and 1.5, between 0.02 and 1.25, between 0.02 and 1, between 0.02 and 0.75, between 0.02 and 0.5, between 0.02 and 0.4, between 0.02 and 0.3, between 0.02 and 0.2, between 0.02 and 0.1, between 0.1 and 0.2, between 0.2 and 0.3, between 0.3 and 0.4, or between 0.2 and 0.5 nM.

29. The method of any of claims 1 to 28, wherein the concentration differences of the excess primer and the limiting primer are optimized for each target sequence.

30. The method of any of claims 1 to 29, wherein the nucleic acid products amplified from the asymmetric PCR reaction differ in their length for individual target sequences.

31. The method of any of claims 1 to 29, wherein the nucleic acid products amplified from the asymmetric PCR reaction have a substantially similar amplicon length.

32. The method of any of claims 1 to 29, wherein the nucleic acid products amplified from the asymmetric PCR reaction have an amplicon length varying by less than 70%, 60%, 50%, 40%, 30%, 20%, 10% or 5%.

33. The method of any of claims 1 to 29, wherein the nucleic acid products amplified from the asymmetric PCR reaction have a length of at least 100000, 75000, 50000, 25000, 10000, 7500, 5000, 2500, 2000, 1500, 1000, 750, 600, 500, 400, 300, 200, 100, 50, 40, 30 or 20 nucleotides.

34. The method of any of claims 1 to 29, wherein the nucleic acid products amplified from the asymmetric PCR reaction have a length of at most 100000, 75000, 50000, 25000, 10000, 7500, 5000, 2500, 2000, 1500, 1000, 750, 600, 500, 400, 300, 200, 100, 50, 40, 30 or 20 nucleotides.

35. The method of any of claims 1 to 29, wherein the nucleic acid products amplified from the asymmetric PCR reaction have a length of between 600 and 500, between 600 and 400, between 600 and 300, between 600 and 200, between 600 and 100, between 600 and 50, between 600 and 40, between 600 and 30, or between 600 and 20 nucleotides.

36. The method of any of claims 1 to 29, wherein the nucleic acid products amplified from the asymmetric PCR reaction have a length of between 600 and 500, between 500 and 400, between 400 and 300, between 300 and 200, between 200 and 100, between 100 and 50, between 100 and 40, between 100 and 30, or between 100 and 20 nucleotides.

37. The method of any of claims 1 to 29, wherein the nucleic acid products amplified from the asymmetric PCR reaction have a length of between 500 and 20, between 400 and 20, between 300 and 20, between 200 and 20, between 150 and 20, between 100 and 20, between 90 and 20, between 80 and 20, between 70 and 20, between 60 and 20, between 50 and 20, between 40 and 20, or between 30 and 20 nucleotides.

38. The method of any of claims 1 to 37, wherein the asymmetric PCR reaction comprises (i) a series of temperature cycles comprising a denaturation step, an annealing step, and an extension step, or (ii) a series of temperature cycles comprising a denaturation step and a combined annealing and extension step.

39. The method of claim 38, wherein the annealing step is at around 55 °C or around 60 °C.

40. The method of claim 38, wherein the annealing step is between 35 and 65, between 40 and 60, between 45 and 55, between 40 and 65, between 45 and 60, between 50 and 60, between 50 and 55, between 55 and 60, or between 60 and 65 °C.

41. The method of any of claims 38 to 40, the extension step is for about 0.5, 1, 2, 3, 4, 5, 6, 7, 8, 9, 10, 11, 12, 13, 15, 18, 20, 30, 40, 50, 60, 80, 100, 120, 140, 160 or 180 minutes.

42. The method of any of claims 38 to 40, the extension step is for at least 0.5, 1, 2, 3, 4, 5, 6, 7, 8, 9, 10, 11, 12, 13, 15, 18, 20, 30, 40, 50, 60, 80, 100, 120, 140, 160 or 180 minutes.

43. The method of any of claims 38 to 40, the extension step is for at most 0.5, 1, 2, 3, 4, 5, 6, 7, 8, 9, 10, 11, 12, 13, 15, 18, 20, 30, 40, 50, 60, 80, 100, 120, 140, 160 or 180 minutes.

44. The method of any of claims 38 to 40, the extension step is for between 0.5 and 20, between 1 and 20, between 2 and 20, between 3 and 20, between 4 and 20, between 5 and 20, between 6 and 20, between 7 and 20, between 8 and 20, between 9 and 20, between 10 and 20, between 11 and 20, between 12 and 20, between 13 and 20, between 15 and 20, between 1 and 18, between 2 and 16, between 3 and 14, between 4 and 12, between 5 and 10, between 6 and 9, between 7 and 8, between 4 and 6, between 6 and 8, between 8 and 10, between 9 and 12, or between 10 and 12 minutes.

45. The method of any of claims 1 to 44, wherein the asymmetric PCR reaction comprises at least about 15, 20, 25, 30, 35, 40, 45, 50, 55, 60, 65, 70, 75, 80, 85 or 90 cycles.

46. The method of any of claims 1 to 44, wherein the asymmetric PCR reaction comprises about 15, 20, 25, 30, 35, 40, 45, 50, 55, 60, 65, 70, 75, 80, 85 or 90 cycles.

47. The method of any of claims 1 to 44, wherein the asymmetric PCR reaction comprises between 15 and 90, between 20 and 80, between 30 and 70, between 40 and 60, between 25 and 90, between 30 and 90, between 40 and 90, between 50 and 90, between 60 and 90, between 70 and 90, between 25 and 80, between 25 and 70, between 25 and 60, between 25 and 50, between 25 and 40, between 15 and 30, between 25 and 50, or between 35 and 50 cycles.

48. The method of any of claims 1 to 47, wherein the excess primer and the limiting primer have different melting temperatures, and the annealing temperature used in the asymmetric PCR reaction is optimized for the excess primer.

49. The method of any of claims 1 to 48, further comprising: normalizing the sequencing reads data or abundance readout by a normalization factor determined based on one or more elements selected from the group consisting of (i) sequencing yield, (ii) sequence-dependent amplification efficiency, (iii) the transform function, and (iv) linearity correction.

50. The method of any of claims 1 to 49, wherein the nucleic acid mixture sample is a cDNA sample.

51. The method of any of claims 1 to 50, wherein the nucleic acid mixture sample is barcoded for cell origin.

52. The method of any of claims 1 to 51, wherein the high-throughput sequencing library is barcoded with unique molecular identifiers (UMIs).

53. A kit comprising a panel of primer pairs, each pair comprising an excess primer and a limiting primer, at a stock concentration configuration pre-determined for asymmetric PCR amplification off a desired target amplicon.

54. The kit of claim 53, the panel comprises at least 10, 20, 50, 100, 200, 300, 400, 500, 750, 1000, 2000, 5000, 10000, 20000, or 100000 primer pairs.

55. The kit of claim 54, wherein subsets, or all, of the primer pairs share the same excess primer.

56. The kit of claim 53 or 54, the excess primer and the limiting primer of each primer pair is packed separately.

57. The kit of claim 53 or 54, the primer pairs are individually pre-mixed.

58. The kit of any of claims 53 to 57, further comprising one or more of the following: a set of dNTPs, a polymerase, a buffer solution, and a set of labeled probes.


SEQUENCE LISTING (44 sequences)

SEQ ID NO: 1 - DNA, 72 bp, synthetic construct
  source 1..72: mol_type = other DNA
gaccccaaaatcagcgaaatgcaccccgcattacgtttggtggaccctcagattcaactggcagtaaccaga

SEQ ID NO: 2 - DNA, 20 bp, synthetic construct
  source 1..20: mol_type = other DNA
gaccccaaaatcagcgaaat

SEQ ID NO: 3 - DNA, 24 bp, synthetic construct
  source 1..24: mol_type = other DNA
tctggttactgccagttgaatctg

SEQ ID NO: 4 - DNA, 24 bp, synthetic construct
  source 1..24: mol_type = other DNA
accccgcattacgtttggtggacc

SEQ ID NO: 5 - DNA, 103 bp, synthetic construct
  source 1..103: mol_type = other DNA
tcctcaactcactcttcgagcgtctcaatgaaggacattcaaagccaattcgagcagctgaaactgcggtgggagtcttatcccaatttggtcaagagcaccg

SEQ ID NO: 6 - DNA, 22 bp, synthetic construct
  source 1..22: mol_type = other DNA
tcctcaactcactcttcgagcg

SEQ ID NO: 7 - DNA, 21 bp, synthetic construct
  source 1..21: mol_type = other DNA
cggtgctcttgaccaaattgg

SEQ ID NO: 8 - DNA, 27 bp, synthetic construct
  source 1..27: mol_type = other DNA
ccaattcgagcagctgaaactgcggtg

SEQ ID NO: 9 - DNA, 92 bp, synthetic construct
  source 1..92: mol_type = other DNA
ctgcagatttggatgatttctccaaacaattgcaacaatccatgagcagtgctgactcaactcaggcctaaactcatgcagaccacacaagg

SEQ ID NO: 10 - DNA, 23 bp, synthetic construct
  source 1..23: mol_type = other DNA
ctgcagatttggatgatttctcc

SEQ ID NO: 11 - DNA, 25 bp, synthetic construct
  source 1..25: mol_type = other DNA
ccttgtgtggtctgcatgagtttag

SEQ ID NO: 12 - DNA, 30 bp, synthetic construct
  source 1..30: mol_type = other DNA
attgcaacaatccatgagcagtgctgactc

SEQ ID NO: 13 - DNA, 65 bp, synthetic construct
  source 1..65: mol_type = other DNA
agatttggacctgcgagcgggttctgacctgaaggctctgcgcggacttgtggagacagccgctc

SEQ ID NO: 14 - DNA, 19 bp, synthetic construct
  source 1..19: mol_type = other DNA
agatttggacctgcgagcg

SEQ ID NO: 15 - DNA, 20 bp, synthetic construct
  source 1..20: mol_type = other DNA
gagcggctgtctccacaagt

SEQ ID NO: 16 - DNA, 23 bp, synthetic construct
  source 1..23: mol_type = other DNA
ttctgacctgaaggctctgcgcg

SEQ ID NO: 17 - DNA, 67 bp, synthetic construct
  source 1..67: mol_type = other DNA
ttacaaacattggccgcaaattgcacaatttgcccccagcgcttcagcgttcttcggaatgtcgcgc

SEQ ID NO: 18 - DNA, 20 bp, synthetic construct
  source 1..20: mol_type = other DNA
ttacaaacattggccgcaaa

SEQ ID NO: 19 - DNA, 18 bp, synthetic construct
  source 1..18: mol_type = other DNA
gcgcgacattccgaagaa

SEQ ID NO: 20 - DNA, 23 bp, synthetic construct
  source 1..23: mol_type = other DNA
acaatttgcccccagcgcttcag

SEQ ID NO: 21 - DNA, 112 bp, synthetic construct
  source 1..112: mol_type = other DNA
gatctgctgtttcactttgggttaaaatcttttgctttcatattgagcttaagagttcagaacactgatggaaaaaacttggctctttgttatttttcagaaatgtattttt

SEQ ID NO: 22 - DNA, 28 bp, synthetic construct
  source 1..28: mol_type = other DNA
atttctgaaaaataacaaagagccaagt

SEQ ID NO: 23 - DNA, 23 bp, synthetic construct
  source 1..23: mol_type = other DNA
tgctgtttcactttgggttaaaa

SEQ ID NO: 24 - DNA, 116 bp, synthetic construct
  source 1..116: mol_type = other DNA
gccattatttgtatcatgggtagcttgatcctcagagcaccttgcaggctgctttctagagtaaggattagaagattagaaatatgtgaacaaactccctcctctacacgctttga

SEQ ID NO: 25 - DNA, 19 bp, synthetic construct
  source 1..19: mol_type = other DNA
tcaaagcgtgtagaggagg

SEQ ID NO: 26 - DNA, 24 bp, synthetic construct
  source 1..24: mol_type = other DNA
gccattatttgtatcatgggtagc

SEQ ID NO: 27 - DNA, 101 bp, synthetic construct
  source 1..101: mol_type = other DNA
ttggctcccaacagtgaactcagctacatgcattcatttagagaataaaagttaatccgggtttggaatccattgcatttgttttgggcacactggtgtct

SEQ ID NO: 28 - DNA, 20 bp, synthetic construct
  source 1..20: mol_type = other DNA
accagtgtgcccaaaacaaa

SEQ ID NO: 29 - DNA, 19 bp, synthetic construct
  source 1..19: mol_type = other DNA
ttggctcccaacagtgaac

SEQ ID NO: 30 - DNA, 115 bp, synthetic construct
  source 1..115: mol_type = other DNA
ccacgctgaatacttctttctaatacacattgaatcgactaccaatatggtgttttggaaaagcttaccactccaatttcatcacaagtctgtatagaccacattacttacaaaa

SEQ ID NO: 31 - DNA, 28 bp, synthetic construct
  source 1..28: mol_type = other DNA
cacgctgaatacttctttctaatacaca

SEQ ID NO: 32 - DNA, 28 bp, synthetic construct
  source 1..28: mol_type = other DNA
ttttgtaagtaatgtggtctatacagac

SEQ ID NO: 33 - DNA, 114 bp, synthetic construct
  source 1..114: mol_type = other DNA
cccaaagtgttgggattacagtcatgagccaccacacctggcccatcataactattttcgagtttgcatggaacccactttgctcccagctttctgcgctggttgcccttaggc

SEQ ID NO: 34 - DNA, 20 bp, synthetic construct
  source 1..20: mol_type = other DNA
gcagaaagctgggagcaaag

SEQ ID NO: 35 - DNA, 22 bp, synthetic construct
  source 1..22: mol_type = other DNA
cccaaagtgttgggattacagt

SEQ ID NO: 36 - DNA, 108 bp, synthetic construct
  source 1..108: mol_type = other DNA
tgtaaatctgttcttctcagttgaaaggataaaagctaaataggaattgcatgaaagctaaataggatttttttctgtcatcgtttattatcacaattatcctcccca

SEQ ID NO: 37 - DNA, 27 bp, synthetic construct
  source 1..27: mol_type = other DNA
tggggaggataattgtgataataaacg

SEQ ID NO: 38 - DNA, 28 bp, synthetic construct
  source 1..28: mol_type = other DNA
agttgaaaggataaaagctaaataggaa

SEQ ID NO: 39 - DNA, 103 bp, synthetic construct
  source 1..103: mol_type = other DNA
tggcgcttgctaaagagcatttaactcatcctgattcaccgcaggattgggctggctttcaaactcctgacagtgaattactgctcttgtaatgacgaatcca

SEQ ID NO: 40 - DNA, 18 bp, synthetic construct
  source 1..18: mol_type = other DNA
ggcgcttgctaaagagca

SEQ ID NO: 41 - DNA, 24 bp, synthetic construct
  source 1..24: mol_type = other DNA
tggattcgtcattacaagagcagt

SEQ ID NO: 42 - DNA, 100 bp, synthetic construct
  source 1..100: mol_type = other DNA
tagcaatattattgtacatatcacagggtatacctgtgtgagagtttcttttgcgtatatatcgagatggaaattgctcatagagtaggtgaatacttga

SEQ ID NO: 43 - DNA, 28 bp, synthetic construct
  source 1..28: mol_type = other DNA
tcaagtattcacctactctatgagcaat

SEQ ID NO: 44 - DNA, 29 bp, synthetic construct
  source 1..29: mol_type = other DNA
tagcaatattattgtacatatcacagggt